(19) United States
(12) Patent Application Publication
(10) Pub. No.: US 2013/0236917 A1
(43) Pub. Date: Sep. 12, 2013

(54) ALBUMIN-BOUND PROTEIN/PEPTIDE COMPLEX AS A BIOMARKER FOR DISEASE

(71) Applicants: Jennifer E. Van Eyk, Baltimore, MD (US); Ronald Joseph Holewinski, Baltimore, MD (US)

(72) Inventors: Jennifer E. Van Eyk, Baltimore, MD (US); Ronald Joseph Holewinski, Baltimore, MD (US)

(73) Assignee: THE JOHNS HOPKINS UNIVERSITY, Baltimore, MD (US)

(21) Appl. No.: 13/885,144

(22) PCT Filed: Nov. 14, 2011
(86) PCT No.: PCT/US11/60642

§ 371 Date: May 13, 2013

Related U.S. Application Data
(60) Provisional application No. 61/412,931, filed on Nov. 12, 2010.

(51) Int. Cl. G01N 33/68

(52) U.S. Cl. CPC G01N 33/6893
USPC 435/7.92; 435/23; 436/501

(57) ABSTRACT

Methods and kits provide for diagnosis and prognosis of ischemia by using biomarkers comprising albumin-bound protein/peptide complex (ABPPC).

### CROSS-REFERENCE TO RELATED APPLICATIONS

This application claims priority to U.S. Provisional Application No. 61/412,931 filed Nov. 12, 2010, the entire contents of which are hereby incorporated by reference.

### STATEMENT OF GOVERNMENT RIGHTS

This invention was made with Government support of an NHLBI proteomic grant, awarded by the National Institutes of Health. The Government has certain rights in this invention.

### FIELD OF INVENTION

The invention relates to methods of diagnosis using biomarkers comprising unique albumin-bound protein/peptide complex(es) (ABPPC).

### BACKGROUND

Serum albumin is the most abundant protein in serum and plasma, typically present at 45-50 mg/ml. Albumin functions as a “molecular sponge” binding proteins, lipids, and small molecules in the intracellular space (Millea, K., Krull, I. Journal of Liquid Chromatography and Related Technologies 2003, 26, 2195-2224; Anderson, N. L., Anderson, N. G. Mol Cell Proteomics 2002, 1, 845-867; Carter, D. C., Ho, J. X. Adv Protein Chem 1994, 45, 153-203) and has been found to form associations with peptide hormones, serum amyloid A, interferons, glucagons, bradykinin, insulin, and Streptococcal Protein G (Peters, T., Jr. All About Albumin; Academic Press San Diego, 1996; Baczynskyj, L., Bronson, G. E., Kubiak, T. M. Rapid Commun Mass Spectrom 1994, 8, 280-286; Carter, W. A. Methods Enzymol 1981, 78, 576-582; Sjobring, U., Bjorck, L., Kastern, W. J Biol Chem 1991, 266, 399-405) but an extensive list of binding partners, and whether these partners change with disease, has not been investigated. Previous studies have shown a higher recovery of low molecular weight species when removing high molecular weight species under denaturing conditions, further confirming that larger proteins, such as albumin, are binding peptides (Tirumalai, R. S., Chan, K. C., Prieto, D. A., Issaq, H. J., Conrads, T. P., Veenstra, T. D. Mol Cell Proteomics 2003, 2, 1096-1103). Furthermore, albumin has been reported to bind to a small number of specific proteins such as paraoxonase 1 (Ortigoza-Ferado, J., Richter, R. J., Hornung, S. K., Motulsky, A. G., Furlong, C. E. Am J Hum Genet 1984, 36, 295-305), alpha-1-acid glycoprotein (Krauss, E., Polnaszek, C. F., Scheeler, D. A., Halsall, H. B., Eckfeldt, J. H., Holtzman, J. L. J Pharmacol Exp Ther 1986, 239, 754-759), and clusterin (Kelso, G. J., Stuart, W. D., Richter, R. J., Furlong, C. E., Jordan-Starck, T. C., Harmony, J. A. Biochemistry 1994, 33, 832-839) (indirect interaction through paraoxonase 1) and apolipoprotein E in serum. Although albumin binding peptides (below 30 kDa) in serum have been studied, the extent of their binding is currently unknown (Zhou, M., Lucas, D. A., Chan, K. C.; Issaq, H. J., Petricoin, E. F., 3^{rd}, Liotta, L. A., Veenstra, T. D., Conrads, T. P. Electrophoresis 2004, 25, 1289-1298). To date, a comprehensive study of the proteins/peptides bound to albumin in ischemic disease has not been carried out.

Albumin has been found to change with disease which alters its binding to metals and currently functions as a biomarker for ischemia. A modification of albumin that has previously been identified as a biomarker for myocardial ischemia is the N-terminus N-acetylation of albumin, which decreases the binding affinity of albumin for cobalt and nickel (Bar-Or, D., Curtis, G., Rao, N., Bampos, N., Lau, E. Eur J Biochem 2001, 268, 42-47; Takahashi, N., Takahashi, Y., Putnam, F. W. Proc Natl Acad Sci USA 1987, 84, 7403-7407; Chan, B., Dodsworth, N., Woodrow, J., Tucker, A., Harris, R. Eur J Biochem 1995, 227, 524-528). Current patents applications (Crosby, P. A. M., Deborah L in PCT Int. Appl: USA, 2002; Bar-or, D. L., Edward; Winkler, James V In PCT Int: US, 2004) disclose the usage of this N-terminal modification of albumin for ischemia and have led to a clinical assay for albumin cobalt binding (ACB assay). In addition to the N-terminal modification, the oxidation of albumin has been proposed to be a marker for oxidative stress (Mera, K., Anraku, M., Kitamura, K., Nakajou, K., Maruyama, T., Tomita, K., Otagiri, M. Hypertens Res 2005, 28, 973-980). MALDI-TOF analysis (Matrix Assisted Laser Desorption/Ionization Time-of-Flight) of the albumin in patients with renal impairment and end-stage renal disease show an increase in the molecular weight (MW) of albumin with disease (Thornalley, P. J., Argirova, M., Ahmed, N., Mann, V. M., Argirov, O., Dawnay, A. Kidney Int 2000, 58, 2228-2234). Finally, the fatty acid transport function of albumin is modified in atherosclerosis and diabetes (Murayskaya, E. V., Lapko, A. G., Murayskii, V. A. Bull Exp Biol Med 2003, 135, 433-435). In patients with diabetes, the binding capacity of albumin for fatty acids is increased, and in patients with atherosclerosis the capacity is decreased. In conclusion, the evidence that albumin is changing with disease is clear. The altered binding of albumin with particular protein/peptide complexes (ABPPC) in ischemic disease has not been identified. Identification of such novel ABPPC complexes in ischemic disease will result in new biomarkers for methods of diagnosing ischemic disease.

Altered binding of proteins and/or peptides to albumin in serum or plasma or other body fluids in ischemic events has not been used to diagnose ischemic disease. The current work is unique because it includes the analysis of intact proteins, degraded proteins, and peptides, without eliminating any mass range in patients with ischemia. Furthermore, the current work focuses on the changes in the proteins and peptides that bind to albumin, in an ischemic disease state.

### SUMMARY

A method of diagnosing ischemia is provided, comprising determining the level of specific albumin-bound protein/peptide complex(es) (ABPPC) in a subject suspected of having ischemia, and quantifying the level determined to a control level from a normal subject population. It has been found that variations in the levels of specific ABPPCs, and variations in ABPPC profile are indicative ischemia.

### BRIEF DESCRIPTION OF THE DRAWINGS

FIG. 1. Size exclusion chromatograms for standard proteins with molecular weights and retention times (in minutes) listed in the table. The red trace if for an albuminome sample taken from a control patient at baseline.

FIG. 2. Size exclusion chromatograms of the ABPPC for patients undergoing PTCA.

FIG. 3. One-dimensional SDS-PAGE for SEC fractions of albuminome taken from control and diseased patients.

FIG. 4. A) Comparison of log 10 spectral counts for proteins in control and diseased group at time-point 1, baseline. B) Comparison of log 10 spectral counts for proteins in control and diseased group at time-point 8, 24 hr post PTCA. Analysis was run using the Stata software.

### DETAILED DESCRIPTION

We examined an albumin-enriched fraction of human serum in order to determine the albumin binding proteins in healthy and diseased individuals.

Accordingly, a method of diagnosing ischemia is provided, comprising determining the level of specific albumin-bound protein/peptide complex(es) (ABPPC) in a subject suspected of having myocardial ischemia, and quantifying the level determined to a control level from a normal subject population. It has been found that variations in the levels of specific ABPPCs, and variations in ABPPC profile are indicative ischemia.

The aim is to characterize proteins/protein fragments/peptides that are differentially bound to albumin in ischemic and healthy patients in a cost effective, rapid and sensitive manner that is compatible with current blood collection protocols. This is based on the hypothesis that albumin changes with disease, and therefore the complex of albumin with its bound proteins and peptides changes, although the inventors are not bound by any particular hypothesis. The ABPPC assay may measure a modification of albumin or a change in ABPPC composition (i.e. the presence or absence of one or more proteins), altered concentration (or stoichiomery or molar ratio) of one or more proteins, change in a protein's PTM (postranslational modification) (e.g. proteolysis fragment vs. intact protein including albumin). The post-translational modification can include oxidation, citrullination, phosphorylation and glycosylation.

Findings have shown that the ABPPC is altered in patients with myocardial ischemia (prior to cell necrosis) and with myocardial infarction and the ABPPC differs in patients with vasculitis and those with ischemia, myocardial infarction and healthy individuals. However, the actual proteins and peptides involved have not been previously identified. Identification of the actual proteins and peptides will improve diagnosis of ischemia by assaying for albumin-bound protein/peptide complex(es) with particular proteins/peptides in mind. Herein lies the advancement in the field of ischemia diagnostics.

The inventors have analyzed the ABPPC obtained from patients with stable angina (SA, control group) and patients with myocardial necrosis or myocardial infarction (MI, diseases group, based on cell necrosis and detection of cTnI or cTnT in blood) who underwent angioplasty (inducing a degree of myocardial ischemia). The ABPPC proteins were quantified using mass spectrometry. The total spectral counts was determined and compared between the SA and MI patients. Certain proteins or peptides increase or decrease in the MI patients compared to the SA patients and these proteins are potential biomarkers for ischemic as well as non-ischemic diseases that change the ABPPC. The findings appear in Table 1.

TABLE 1
Proteins detected in the albumin-binding protein/peptide complex

| | | | | | | | ISCHEMIA - | | |
|---|---|---|---|---|---|---|---|---|---|
| | | | | SA - Average | | | Average Spectral | | |
| | Accession | | | Spectral Count | | | count | | |
| Protein # | Number | Protein Name | MW | TP1 | TP7 | TP8 | TP1 | TP7 | TP8 |
| 1 | IPI00027462 | Protein S100-A9 # | 13 kDa | 20.0 | 31.7 | 29.3 | 31.0 | 19.0 | 75.7 |
| 3 | IPI00007047 | Protein S100-A8 # | 11 kDa | 11.3 | 14.3 | 16.3 | 15.0 | 9.7 | 34.0 |
| 4 | IPI00025753 | Desmoglein-1 # | 114 kDa | 13.7 | 14.3 | 12.3 | 12.7 | 13.7 | 25.7 |
| 5 | IPI00795257 | Glyceraldehyde-3- | 32 kDa | 6.3 | 9.0 | 7.7 | 10.0 | 4.7 | 31.7 |
| | | phosphate Dehydrogenase # | | | | | | | |
| 6 | IPI00219806 | Protein S100-A7 # | 11 kDa | 8.3 | 8.7 | 9.0 | 9.0 | 8.7 | 25.3 |
| 7 | IPI00455315 | Annexin A2 # | 39 kDa | 3.3 | 10.2 | 2.2 | 6.8 | 4.3 | 29.5 |
| 8 | IPI00554711 | Plakoglobin # | 82 kDa | 1.2 | 6.8 | 1.8 | 5.3 | 1.0 | 22.0 |
| 9 | IPI00031564 | Gamma- | 21 kDa | 1.3 | 1.5 | 0.7 | 2.5 | 4.0 | 6.0 |
| | | glutamylcyclotransferase* @ | | | | | | | |
| 10 | IPI00017987 | Cornifin-A @ | 10 kDa | 0.5 | 0.5 | 1.2 | 0.8 | 2.0 | 4.3 |
| 11 | IPI00000874 | Peroxiredoxin-1 # | 22 kDa | 0.5 | 0.5 | 0.7 | 1.0 | 0.5 | 3.2 |
| 12 | IPI00646687 | Protein POF1B* # | 68 kDa | 0.5 | 0.5 | 0.5 | 1.3 | 0.5 | 5.8 |
| 13 | IPI00218528 | Plakophilin-1* # | 80 kDa | 0.5 | 0.5 | 0.5 | 0.5 | 0.5 | 4.2 |
| 14 | IPI00162735 | Attractin* α | 141 kDa | 3.0 | 4.0 | 7.7 | 5.5 | 8.5 | 9.5 |
| 15 | IPI00465436 | Catalase # | 60 kDa | 0.7 | 0.5 | 0.7 | 0.5 | 0.5 | 2.5 |
| 17 | IPI00022463 | Serotransferrin | 77 kDa | 54.3 | 67.0 | 73.3 | 74.7 | 74.0 | 59.3 |
| 20 | IPI00784985 | IGK@ protein | 26 kDa | 18.0 | 31.0 | 44.7 | 36.3 | 30.0 | 46.3 |
| 22 | IPI00013885 | Caspase-14 # | 28 kDa | 21.0 | 22.0 | 19.3 | 24.3 | 29.7 | 40.0 |
| 23 | IPI00478003 | Alpha-2-macroglobulin | 163 kDa | 36.8 | 28.3 | 36.7 | 5.0 | 18.2 | 31.2 |
| 25 | IPI00021440 | Actin, cytoplasmic 2 % | 42 kDa | 6.0 | 18.0 | 6.8 | 17.7 | 5.8 | 34.3 |
| 26 | IPI00009650 | Lipocalin-1 | 19 kDa | 15.0 | 16.0 | 15.3 | 17.0 | 16.3 | 12.3 |
| 27 | IPI00978930 | Ig alpha-1 chain C region | 53 kDa | 9.0 | 14.7 | 19.0 | 14.7 | 16.0 | 18.3 |
| 29 | IPI00019038 | Lysozyme C | 17 kDa | 10.3 | 7.7 | 11.3 | 16.3 | 12.0 | 12.3 |
| 30 | IPI00032325 | Cystatin-A | 11 kDa | 11.7 | 13.3 | 11.3 | 11.3 | 13.0 | 13.0 |
| 31 | IPI00022204 | Serine protease inhibitor | 45 kDa | 3.3 | 9.0 | 3.7 | 11.7 | 3.7 | 34.0 |
| | | B3* % | | | | | | | |
| 32 | IPI00027547 | Dermcidin | 11 kDa | 7.3 | 11.3 | 12.3 | 12.0 | 13.7 | 9.3 |
| 36 | IPI00456429 | Ubiquitin and ribosomal | 15 kDa | 10.3 | 11.0 | 11.7 | 10.7 | 10.3 | 11.0 |
| | | protein L40 precursor | | | | | | | |
| 37 | IPI00397801 | Filaggrin-2 | 248 kDa | 9.7 | 11.3 | 12.0 | 8.7 | 9.0 | 14.3 |
| 38 | IPI00022974 | Prolactin-inducible protein | 17 kDa | 10.7 | 10.7 | 10.7 | 8.7 | 10.3 | 8.3 |
| 39 | IPI00423463 | Putative uncharacterized | 53 kDa | 1.7 | 2.8 | 12.7 | 11.0 | 7.0 | 24.3 |
| | | protein | | | | | | | |
| | | DKFZp686O01196 @ | | | | | | | |
| 42 | IPI00007797 | Fatty acid-binding protein, | 15 kDa | 3.0 | 4.3 | 4.0 | 4.3 | 4.3 | 5.3 |
| | | epidermal | | | | | | | |
| 44 | IPI00871372 | E3 ubiquitin-protein ligase | 289 kDa | 4.0 | 1.5 | 2.0 | 2.0 | 2.7 | 2.0 |
| | | HECTD1 | | | | | | | |
| 45 | IPI00021536 | Calmodulin-like protein 5 # | 16 kDa | 4.5 | 5.7 | 2.2 | 7.7 | 3.7 | 13.5 |
| 46 | IPI00219221 | Galectin-7 # | 15 kDa | 1.8 | 6.7 | 2.3 | 6.3 | 4.3 | 12.3 |
| 47 | IPI00300376 | Protein-glutamine | 77 kDa | 4.3 | 3.7 | 5.0 | 5.2 | 7.3 | 5.7 |
| | | gamma- | | | | | | | |
| | | glutamyltransferase E α | | | | | | | |
| 48 | IPI00552749 | Dynein heavy chain 8, | 478 kDa | 3.0 | 3.0 | 3.2 | 6.2 | 4.2 | 6.2 |
| | | axonemal | | | | | | | |
| 49 | IPI00396485 | Elongation factor 1-alpha | 50 kDa | 4.5 | 6.0 | 1.0 | 6.0 | 1.7 | 11.0 |
| | | 1% | | | | | | | |
| 50 | IPI00219217 | L-lactate dehydrogenase | 37 kDa | 3.5 | 3.3 | 3.0 | 6.2 | 6.7 | 7.5 |
| | | B chain @ | | | | | | | |
| 51 | IPI00006662 | Apolipoprotein D α | 21 kDa | 1.2 | 2.7 | 2.0 | 1.2 | 6.3 | 2.3 |
| 52 | IPI00291866 | Plasma protease C1 | 55 kDa | 0.7 | 3.8 | 4.7 | 1.0 | 1.7 | 0.8 |
| | | inhibitor β | | | | | | | |
| 53 | IPI00216298 | Thioredoxin | 12 kDa | 4.0 | 3.3 | 5.0 | 3.3 | 5.3 | 5.0 |
| 55 | IPI00908330 | Excitatory amino acid | 54 kDa | 2.8 | 2.7 | 1.7 | 2.5 | 3.7 | 2.0 |
| | | transporter 1 | | | | | | | |
| 56 | IPI00643202 | SERPINB12 protein | 48 kDa | 3.7 | 4.0 | 3.0 | 2.2 | 3.5 | 5.0 |
| 57 | IPI00453473 | Histone H4 % | 11 kDa | 1.3 | 5.5 | 2.7 | 4.5 | 0.5 | 9.7 |
| 58 | IPI00008290 | Ephrin type-A receptor 5 α | 115 kDa | 0.5 | 0.7 | 1.5 | 5.2 | 4.8 | 2.2 |
| 59 | IPI00903112 | Lactotransferrin δ | 77 kDa | 0.8 | 0.5 | 16.8 | 1.5 | 1.0 | 0.8 |
| 61 | IPI00383347 | PRO2194 α | 14 kDa | 0.8 | 1.5 | 2.0 | 2.3 | 3.3 | 2.3 |
| 62 | IPI00154742 | IGL@ protein | 25 kDa | 1.3 | 2.0 | 2.8 | 2.5 | 3.2 | 5.0 |
| 65 | IPI00019502 | Myosin-9* # | 227 kDa | 0.5 | 0.5 | 0.5 | 1.0 | 0.5 | 15.7 |
| 66 | IPI00011692 | Involucrin # | 70 kDa | 0.5 | 0.5 | 0.5 | 4.3 | 0.5 | 12.3 |
| 67 | IPI00218343 | Tubulin alpha-1C chain # | 50 kDa | 0.5 | 0.7 | 0.5 | 1.3 | 0.5 | 12.3 |
| 68 | IPI00411765 | 14-3-3 protein sigma* % | 24 kDa | 0.5 | 4.0 | 0.5 | 5.7 | 1.3 | 5.8 |
| 69 | IPI00026256 | Filaggrin # | 435 kDa | 0.5 | 2.2 | 0.5 | 2.0 | 1.2 | 7.3 |
| 70 | IPI00019884 | Alpha-actinin-2 % | 104 kDa | 0.5 | 12.3 | 0.5 | 0.5 | 0.5 | 2.3 |
| 71 | IPI00303476 | ATP synthase subunit | 57 kDa | 0.7 | 8.7 | 0.5 | 0.5 | 0.5 | 5.3 |
| | | beta, mitochondrial % | | | | | | | |
| 72 | IPI00008964 | Ras-related protein Rab- | 22 kDa | 0.5 | 0.5 | 0.5 | 5.7 | 0.5 | 8.3 |
| | | 1B # | | | | | | | |
| 74 | IPI00291560 | Arginase-1* % | 35 kDa | 0.5 | 2.0 | 0.5 | 0.5 | 0.5 | 10.2 |
| 75 | IPI00217966 | L-lactate dehydrogenase # | 40 kDa | 0.5 | 0.7 | 0.5 | 3.3 | 0.5 | 9.3 |
| 77 | IPI00291467 | ADP/ATP translocase 3 % | 33 kDa | 1.5 | 4.0 | 0.7 | 0.5 | 1.0 | 3.8 |
| 78 | IPI00514201 | Myosin-6 γ | 224 kDa | 4.3 | 8.0 | 0.5 | 0.5 | 0.5 | 0.5 |
| 79 | IPI00025512 | Heat shock protein beta-1 % | 23 kDa | 0.5 | 1.8 | 0.5 | 2.0 | 0.5 | 4.3 |
| 80 | IPI00216984 | Calmodulin-like protein 3 @ | 17 kDa | 0.5 | 0.5 | 0.5 | 3.7 | 2.0 | 6.3 |
| 81 | IPI00013895 | Protein S100-A11 % | 12 kDa | 0.5 | 1.7 | 0.7 | 3.0 | 0.5 | 6.0 |
| 82 | IPI00011654 | Tubulin beta chain # | 50 kDa | 0.5 | 0.5 | 0.5 | 0.5 | 0.5 | 11.0 |
| 83 | IPI00032294 | Cystatin-S # | 16 kDa | 1.2 | 2.5 | 0.7 | 1.5 | 2.2 | 1.5 |
| 84 | IPI00908963 | ATP synthase subunit | 58 kDa | 0.5 | 6.7 | 0.5 | 0.5 | 0.5 | 4.7 |
| | | alpha % | | | | | | | |
| 85 | IPI00479186 | Pyruvate kinase isozymes | 58 kDa | 0.7 | 1.3 | 0.7 | 1.3 | 0.5 | 6.0 |
| | | M1/M2* # | | | | | | | |
| 86 | IPI00216952 | Lamin-A/C - (Progerin)* # | 65 kDa | 0.5 | 0.5 | 0.7 | 1.0 | 0.5 | 7.5 |
| 87 | IPI00304621 | Zinc finger protein 518B # | 120 kDa | 0.5 | 0.5 | 0.7 | 1.2 | 2.0 | 2.2 |
| 88 | IPI00218918 | Annexin A1 % | 39 kDa | 0.5 | 4.5 | 0.8 | 0.7 | 2.0 | 2.3 |
| 89 | IPI00419215 | Alpha-2-macroglobulin- | 161 kDa | 0.5 | 0.5 | 0.7 | 0.5 | 0.5 | 8.8 |
| | | like protein 1 # | | | | | | | |
| 90 | IPI00011229 | Cathepsin D # | 45 kDa | 0.5 | 1.0 | 1.0 | 1.3 | 1.3 | 5.2 |
| 91 | IPI00796333 | Fructose-bisphosphate | 45 kDa | 0.5 | 2.2 | 0.5 | 3.0 | 0.5 | 2.3 |
| | | aldolase A % | | | | | | | |
| 92 | IPI00020101 | Histone H2B % | 14 kDa | 0.5 | 1.5 | 1.0 | 0.5 | 0.5 | 7.0 |
| 93 | IPI00004573 | Polymeric immunoglobulin | 83 kDa | 0.5 | 0.5 | 6.7 | 0.5 | 1.3 | 0.5 |
| | | receptor δ | | | | | | | |
| 94 | IPI00386975 | Desmocollin-1* | 94 kDa | 1.5 | 1.0 | 1.3 | 0.5 | 1.5 | 1.7 |
| 95 | IPI00022426 | Protein AMBP | 39 kDa | 0.5 | 2.0 | 3.0 | 0.5 | 1.5 | 2.8 |
| 96 | IPI00017992 | Small proline-rich protein | 8 kDa | 2.0 | 0.7 | 0.5 | 2.2 | 1.5 | 1.0 |
| | | 2B @ | | | | | | | |
| 97 | IPI00023006 | Actin, alpha cardiac | 42 kDa | 0.5 | 2.8 | 1.3 | 1.3 | 0.5 | 3.0 |
| | | muscle 1 % | | | | | | | |
| 98 | IPI00930072 | Putative uncharacterized | 52 kDa | 0.5 | 0.5 | 2.3 | 1.3 | 0.7 | 3.8 |
| | | protein DKFZp686E23209 | | | | | | | |
| 99 | IPI00465248 | Alpha-enolase* # | 47 kDa | 0.5 | 0.7 | 0.5 | 2.7 | 0.5 | 3.7 |
| 100 | IPI00414676 | Heat shock protein HSP | 83 kDa | 0.5 | 0.5 | 0.5 | 1.7 | 0.5 | 6.0 |
| | | 90-beta # | | | | | | | |
| 101 | IPI00296039 | Tropomyosin alpha-1 | 33 kDa | 0.5 | 5.7 | 0.5 | 0.5 | 0.5 | 1.0 |
| | | chain* γ | | | | | | | |
| 102 | IPI00909570 | Elongation factor 2 # | 63 kDa | 0.5 | 0.5 | 0.5 | 1.0 | 0.5 | 6.0 |
| 103 | IPI00013808 | Alpha-actinin-4 # | 105 kDa | 0.5 | 0.5 | 0.5 | 1.7 | 0.5 | 5.0 |
| 104 | IPI00643623 | Neutrophil gelatinase- | 23 kDa | 0.5 | 0.5 | 2.0 | 0.5 | 0.5 | 5.0 |
| | | associated lipocalin # | | | | | | | |
| 105 | IPI00305622 | Protein-glutamine | 90 kDa | 0.5 | 0.5 | 0.5 | 0.5 | 0.5 | 6.0 |
| | | gamma- | | | | | | | |
| | | glutamyltransferase K # | | | | | | | |
| 106 | IPI00216798 | Myosin regulatory light | 19 kDa | 0.5 | 5.7 | 0.5 | 0.5 | 0.5 | 0.5 |
| | | chain 2, ventricular/cardiac | | | | | | | |
| | | muscle isoform* γ | | | | | | | |
| 107 | IPI00335168 | Isoform Non-muscle of | 17 kDa | 0.5 | 2.3 | 0.5 | 0.5 | 0.5 | 2.7 |
| | | Myosin light polypeptide | | | | | | | |
| | | 6* % | | | | | | | |
| 108 | IPI00790739 | Aconitase 2, mitochondrial γ | 88 kDa | 0.5 | 6.3 | 0.5 | 0.5 | 0.5 | 0.5 |
| 109 | IPI00848226 | Guanine nucleotide- | 35 kDa | 0.5 | 0.5 | 0.5 | 0.5 | 0.5 | 6.0 |
| | | binding protein subunit | | | | | | | |
| | | beta-2-like 1 # | | | | | | | |
| 110 | IPI00003362 | 78 kDa glucose-regulated | 72 kDa | 0.5 | 0.5 | 0.5 | 1.3 | 0.5 | 4.7 |
| | | protein # | | | | | | | |
| 111 | IPI00412407 | Serpin B4 % | 42 kDa | 0.5 | 2.0 | 0.5 | 1.0 | 0.5 | 3.0 |
| 112 | IPI00877726 | Creatine Kinase type mu, | 50 kDa | 0.5 | 0.5 | 0.5 | 0.5 | 0.5 | 5.0 |
| | | mitochondrial* # | | | | | | | |
| 113 | IPI00426051 | Putative uncharacterized | 51 kDa | 0.5 | 0.5 | 1.0 | 1.7 | 0.5 | 2.7 |
| | | protein | | | | | | | |
| | | DKFZp686C15213 # | | | | | | | |
| 114 | IPI00419585 | Peptidyl-prolyl cis-trans | 18 kDa | 0.5 | 0.7 | 0.5 | 1.3 | 0.5 | 3.3 |
| | | isomerase A # | | | | | | | |
| 115 | IPI00893099 | Heat shock 70 kDa protein | 70 kDa | 0.5 | 0.5 | 0.5 | 1.7 | 0.5 | 3.3 |
| | | 1-like variant # | | | | | | | |
| 116 | IPI00794543 | Calmodulin # | 17 kDa | 0.5 | 0.5 | 0.5 | 1.0 | 0.5 | 4.3 |
| 117 | IPI00219757 | Glutathione S-transferase | 23 kDa | 0.5 | 0.5 | 0.5 | 1.7 | 0.5 | 3.3 |
| | | P # | | | | | | | |
| 118 | IPI00021828 | Cystatin-B % | 11 kDa | 0.5 | 1.7 | 0.5 | 0.5 | 0.5 | 3.0 |
| 119 | IPI00291922 | Proteasome subunit alpha | 26 kDa | 0.5 | 0.5 | 0.5 | 0.5 | 0.5 | 4.0 |
| | | type-5 # | | | | | | | |
| 120 | IPI00021812 | Neuroblast | 629 kDa | 0.5 | 0.5 | 0.5 | 0.5 | 0.5 | 3.3 |
| | | differentiation-associated | | | | | | | |
| | | protein # | | | | | | | |
| 121 | IPI00060800 | Zymogen granule protein | 23 kDa | 0.5 | 0.5 | 2.0 | 2.0 | 0.5 | 0.5 |
| | | 16 homolog B δ | | | | | | | |
| 123 | IPI00291006 | Malate dehydrogenase, | 36 kDa | 0.5 | 2.0 | 0.5 | 0.5 | 0.5 | 1.0 |
| | | mitochondrial % | | | | | | | |
| 124 | IPI00215917 | ADP-ribosylation factor 3 # | 21 kDa | 0.7 | 0.5 | 0.5 | 0.5 | 0.5 | 3.7 |
| 125 | IPI00009856 | Protein Plunc | 27 kDa | 0.5 | 0.5 | 3.7 | 0.5 | 0.5 | 0.7 |
| 126 | IPI00215965 | Heterogeneous nuclear | 39 kDa | 0.5 | 0.5 | 0.5 | 0.5 | 0.5 | 3.3 |
| | | ribonucleoprotein A1* # | | | | | | | |
| 127 | IPI00216026 | Voltage-dependent anion- | 32 kDa | 0.5 | 0.5 | 0.5 | 0.5 | 0.5 | 3.3 |
| | | selective channel protein | | | | | | | |
| | | 2* # | | | | | | | |
| 128 | IPI00873099 | Protein S100A2 # | 11 kDa | 0.5 | 0.5 | 0.5 | 0.5 | 0.5 | 2.3 |
| 129 | IPI00414684 | Semenogelin-1* δ | 45 kDa | 0.5 | 0.5 | 3.7 | 0.5 | 0.5 | 0.5 |
| 130 | IPI00797270 | Triosephosphate | 27 kDa | 0.5 | 1.5 | 0.5 | 1.7 | 0.5 | 1.3 |
| | | isomerase % | | | | | | | |
| 131 | IPI00022990 | Statherin | 7 kDa | 2.0 | 0.5 | 0.5 | 1.7 | 0.5 | 0.5 |
| 132 | IPI00022774 | Transitional endoplasmic | 89 kDa | 0.5 | 0.5 | 0.5 | 0.7 | 0.5 | 2.3 |
| | | reticulum ATPase # | | | | | | | |
| 134 | IPI00879238 | 40S ribosomal protein S9 # | 17 kDa | 0.5 | 0.5 | 0.5 | 0.5 | 0.5 | 2.3 |
| 136 | IPI00216975 | Tropomyosin alpha-4 | 33 kDa | 0.5 | 0.5 | 0.7 | 0.5 | 0.5 | 2.0 |
| | | chain* # | | | | | | | |
| 137 | IPI00232492 | Tripartite motif- | 64 kDa | 0.5 | 0.5 | 0.5 | 0.5 | 0.5 | 3.0 |
| | | containing protein 29* # | | | | | | | |
| 138 | IPI00017672 | Purine nucleoside | 33 kDa | 0.5 | 0.5 | 0.5 | 0.5 | 0.5 | 3.0 |
| | | phosphorylase # | | | | | | | |
| 139 | IPI00007188 | ADP/ATP translocase 2 # | 33 kDa | 0.5 | 0.5 | 0.5 | 0.5 | 0.5 | 3.0 |
| 140 | IPI00243742 | Myosin light chain 3 γ | 22 kDa | 0.5 | 2.7 | 0.5 | 0.5 | 0.5 | 0.5 |
| 141 | IPI00291410 | Long palate, lung and nasal | 52 kDa | 0.5 | 0.5 | 1.7 | 1.0 | 0.7 | 0.5 |
| | | epithelium carcinoma- | | | | | | | |
| | | associated protein 1* δ | | | | | | | |
| 143 | IPI00216691 | Profilin-1δ | 15 kDa | 0.5 | 0.5 | 2.3 | 0.7 | 0.5 | 0.5 |
| 144 | IPI00790304 | Voltage-dependent anion- | 20 kDa | 0.5 | 0.5 | 0.5 | 0.5 | 0.5 | 2.7 |
| | | selective channel protein 1 # | | | | | | | |
| 145 | IPI00015141 | Creatine kinase, sarcomeric | 48 kDa | 0.5 | 2.7 | 0.5 | 0.5 | 0.5 | 0.5 |
| | | mitochondrial γ | | | | | | | |
| 146 | IPI00244346 | Troponin I, cardiac muscle γ | 24 kDa | 0.5 | 2.7 | 0.5 | 0.5 | 0.5 | 0.5 |
| 147 | IPI00556485 | 60S acidic ribosomal | 27 kDa | 0.5 | 0.5 | 0.5 | 0.5 | 0.5 | 2.7 |
| | | protein P0 # | | | | | | | |
| 148 | IPI00012011 | Cofilin-1 # | 19 kDa | 0.5 | 0.5 | 0.5 | 0.7 | 0.5 | 2.0 |
| 149 | IPI00915941 | 60 kDa heat shock | 25 kDa | 0.5 | 0.7 | 0.5 | 0.5 | 0.5 | 1.7 |
| | | protein, mitochondrial # | | | | | | | |
| 150 | IPI00186711 | Plectin-1* # | 518 kDa | 0.5 | 0.5 | 0.7 | 0.5 | 0.5 | 1.7 |
| 151 | IPI00455383 | Clathrin heavy chain 1* # | 188 kDa | 0.5 | 0.5 | 0.5 | 0.5 | 0.5 | 2.0 |
| 152 | IPI00328328 | Eukaryotic initiation | 46 kDa | 0.5 | 0.5 | 0.5 | 0.5 | 0.5 | 2.3 |
| | | factor 4A-II* # | | | | | | | |
| 153 | IPI00479306 | Proteasome subunit beta | 28 kDa | 0.5 | 0.5 | 0.5 | 0.5 | 0.5 | 2.3 |
| | | type-5 # | | | | | | | |
| 154 | IPI00926685 | Tubulin beta-4 chain # | 41 kDa | 0.5 | 0.5 | 0.5 | 0.5 | 0.5 | 2.0 |
| 155 | IPI00010214 | Protein S100-A14 # | 12 kDa | 0.5 | 0.5 | 0.5 | 0.5 | 0.5 | 2.3 |
| 156 | IPI00382482 | Ig heavy chain V-III | 14 kDa | 0.5 | 0.5 | 0.5 | 0.5 | 0.8 | 1.2 |
| | | region CAM # | | | | | | | |
| 157 | IPI00219575 | Bleomycin hydrolase # | 53 kDa | 0.5 | 0.5 | 0.5 | 0.5 | 0.5 | 1.7 |
| 158 | IPI00798035 | Myosin-binding protein C, | 141 kDa | 0.5 | 2.3 | 0.5 | 0.5 | 0.5 | 0.5 |
| | | cardiac-type γ | | | | | | | |
| 159 | IPI00025491 | Eukaryotic initiation | 46 kDa | 0.5 | 0.5 | 0.5 | 0.5 | 0.5 | 1.7 |
| | | factor 4A-I # | | | | | | | |
| 160 | IPI00329389 | 60S ribosomal protein L6# | 33 kDa | 0.5 | 0.5 | 0.5 | 0.5 | 0.5 | 1.7 |
| 161 | IPI00645201 | Ribosomal protein S8 # | 22 kDa | 0.5 | 0.5 | 0.5 | 0.5 | 0.5 | 1.7 |
| 162 | IPI00289983 | Prostatic acid | 48 kDa | 0.5 | 0.5 | 0.5 | 0.5 | 0.5 | 1.3 |
| | | phosphatase* # | | | | | | | |
| 163 | IPI00925023 | NADH-ubiquinone | 74 kDa | 0.5 | 2.0 | 0.5 | 0.5 | 0.5 | 0.5 |
| | | oxidoreductase 75 kDa | | | | | | | |
| | | subunit, mitochondrial γ | | | | | | | |
| 164 | IPI00473011 | Hemoglobin subunit delta # | 16 kDa | 0.5 | 0.7 | 0.5 | 0.5 | 0.5 | 1.7 |
| 165 | IPI00018146 | 14-3-3 protein theta # | 28 kDa | 0.5 | 0.5 | 0.5 | 0.5 | 0.5 | 2.0 |
| 166 | IPI00154509 | Proteasome subunit alpha | 29 kDa | 0.5 | 0.5 | 0.5 | 0.5 | 0.5 | 2.0 |
| | | type-7-like # | | | | | | | |
| 167 | IPI00759776 | Actinin, alpha 1* i # | 106 kDa | 0.5 | 0.5 | 0.5 | 0.5 | 0.5 | 2.0 |
| 168 | IPI00909534 | Elongation factor 1- | 24 kDa | 0.5 | 0.5 | 0.5 | 0.5 | 0.5 | 2.0 |
| | | gamma # | | | | | | | |
| 169 | IPI00414696 | Heterogeneous nuclear | 36 kDa | 0.5 | 0.5 | 0.5 | 0.5 | 0.5 | 1.8 |
| | | ribonucleoproteins | | | | | | | |
| | | A2/B1* # | | | | | | | |
| 170 | IPI00916818 | Phosphoglycerate kinase | 35 kDa | 0.5 | 0.7 | 0.5 | 1.7 | 0.5 | 0.5 |
| 172 | IPI00216318 | 14-3-3 protein beta/alpha* # | 28 kDa | 0.5 | 0.5 | 0.5 | 0.5 | 0.5 | 1.3 |
| 173 | IPI00550363 | Transgelin-2 # | 22 kDa | 0.5 | 0.5 | 0.5 | 0.5 | 0.5 | 1.7 |
| 174 | IPI00301021 | Translocon-associated | 32 kDa | 0.5 | 0.5 | 0.5 | 0.5 | 0.5 | 1.7 |
| | | protein subunit alpha* # | | | | | | | |
| 175 | IPI00871956 | Similar to 40S ribosomal | 20 kDa | 0.5 | 0.5 | 0.5 | 0.5 | 0.5 | 1.7 |
| | | protein S2 # | | | | | | | |
| 177 | IPI00220740 | Nucleophosmin* # | 29 kDa | 0.5 | 0.5 | 0.5 | 0.5 | 0.5 | 1.7 |
| 178 | IPI00023635 | Inositol monophosphatase | 31 kDa | 0.5 | 0.5 | 0.5 | 0.5 | 0.5 | 1.3 |
| | | 2* # | | | | | | | |
| 179 | IPI00031549 | Desmocollin-3* # | 100 kDa | 0.5 | 0.5 | 0.5 | 0.5 | 0.5 | 1.0 |
| 180 | IPI00555956 | Proteasome subunit beta | 29 kDa | 0.5 | 0.5 | 0.5 | 0.5 | 0.5 | 1.0 |
| | | type-4 # | | | | | | | |
| 181 | IPI00478287 | Putative uncharacterized | 22 kDa | 0.5 | 0.5 | 0.5 | 0.5 | 0.5 | 1.3 |
| | | protein | | | | | | | |
| | | ENSP00000352132 # | | | | | | | |
| 182 | IPI00219038 | Histone H3.3 # | 15 kDa | 0.5 | 0.5 | 0.5 | 0.5 | 0.5 | 1.0 |
| 184 | IPI00941747 | Calnexin # | 68 kDa | 0.5 | 0.5 | 0.5 | 0.5 | 0.5 | 1.0 |
| 185 | IPI00219622 | Proteasome subunit alpha | 26 kDa | 0.5 | 0.5 | 0.5 | 0.5 | 0.5 | 1.0 |
| | | type-2 # | | | | | | | |
| 186 | IPI00873506 | Guanine aminohydrolase # | 53 kDa | 0.5 | 0.5 | 0.5 | 0.5 | 0.5 | 1.0 |

Footnote
All isoforms are covered for proteins marked with an asterisk (*)
TP1—Baseline before surgery
TP7—1 hr post PTCA
TP8—24 hr Post PTCA
Proteins in bold are elevated in diseased group at either TP7 or TP8
Proteins in italics are decreased in diseased group based at either TP7 or TP8
# elevated by at least two fold in diseased at TP8 only
@ elevated by at least two fold at in diseased at TP7 and remain elevated at TP8
α—Elevated by at least two fold in diseased at TP7 and return to baseline at TP8
% decreased by at least two fold in diseased at TP7 and increase by at least two fold in diseased at TP8
β—decreased by at least two fold in diseased at TP7 and remain decreased at TP8
γ—decreased by at least two fold in diseased at TP7 and return to baseline at TP8
δ—decreased by at least two fold in diseased at TP8 only

The particular proteins/peptides which are elevated or decreased in the ischemic group appears in Table 2.

TABLE 2
Changes in Proteins in Diseased Individuals

| | | | | | | | ISCHEMIA - | | |
|---|---|---|---|---|---|---|---|---|---|
| | | | | SA - Average | | | Average Spectral | | |
| | Accession | | | Spectral Count | | | count | | |
| Protein # | Number | Protein Name | MW | TP1 | TP7 | TP8 | TP1 | TP7 | TP8 |
| 1 | IPI00027462 | Protein S100-A9 # | 13 kDa | 20.0 | 31.7 | 29.3 | 31.0 | 19.0 | 75.7 |
| 3 | IPI00007047 | Protein S100-A8 # | 11 kDa | 11.3 | 14.3 | 16.3 | 15.0 | 9.7 | 34.0 |
| 4 | IPI00025753 | Desmoglein-1 # | 114 kDa | 13.7 | 14.3 | 12.3 | 12.7 | 13.7 | 25.7 |
| 5 | IPI00795257 | Glyceraldehyde-3- | 32 kDa | 6.3 | 9.0 | 7.7 | 10.0 | 4.7 | 31.7 |
| | | phosphate Dehydrogenase # | | | | | | | |
| 6 | IPI00219806 | Protein S100-A7 # | 11 kDa | 8.3 | 8.7 | 9.0 | 9.0 | 8.7 | 25.3 |
| 7 | IPI00455315 | Annexin A2 # | 39 kDa | 3.3 | 10.2 | 2.2 | 6.8 | 4.3 | 29.5 |
| 8 | IPI00554711 | Plakoglobin # | 82 kDa | 1.2 | 6.8 | 1.8 | 5.3 | 1.0 | 22.0 |
| 9 | IPI00031564 | Gamma- | 21 kDa | 1.3 | 1.5 | 0.7 | 2.5 | 4.0 | 6.0 |
| | | glutamylcyclotransferase* @ | | | | | | | |
| 10 | IPI00017987 | Cornifin-A @ | 10 kDa | 0.5 | 0.5 | 1.2 | 0.8 | 2.0 | 4.3 |
| 11 | IPI00000874 | Peroxiredoxin-1 # | 22 kDa | 0.5 | 0.5 | 0.7 | 1.0 | 0.5 | 3.2 |
| 12 | IPI00646687 | Protein POF1B* # | 68 kDa | 0.5 | 0.5 | 0.5 | 1.3 | 0.5 | 5.8 |
| 13 | IPI00218528 | Plakophilin-1* # | 80 kDa | 0.5 | 0.5 | 0.5 | 0.5 | 0.5 | 4.2 |
| 14 | IPI00162735 | Attractin* α | 141 kDa | 3.0 | 4.0 | 7.7 | 5.5 | 8.5 | 9.5 |
| 15 | IPI00465436 | Catalase # | 60 kDa | 0.7 | 0.5 | 0.7 | 0.5 | 0.5 | 2.5 |
| 22 | IPI00013885 | Caspase-14 # | 28 kDa | 21.0 | 22.0 | 19.3 | 24.3 | 29.7 | 40.0 |
| 25 | IPI00021440 | Actin, cytoplasmic 2 % | 42 kDa | 6.0 | 18.0 | 6.8 | 17.7 | 5.8 | 34.3 |
| 31 | IPI00022204 | Serine protease inhibitor | 45 kDa | 3.3 | 9.0 | 3.7 | 11.7 | 3.7 | 34.0 |
| | | B3* % | | | | | | | |
| 39 | IPI00423463 | Putative uncharacterized | 53 kDa | 1.7 | 2.8 | 12.7 | 11.0 | 7.0 | 24.3 |
| | | protein | | | | | | | |
| | | DKFZp686O01196 @ | | | | | | | |
| 45 | IPI00021536 | Calmodulin-like protein 5 # | 16 kDa | 4.5 | 5.7 | 2.2 | 7.7 | 3.7 | 13.5 |
| 46 | IPI00219221 | Galectin-7 # | 15 kDa | 1.8 | 6.7 | 2.3 | 6.3 | 4.3 | 12.3 |
| 47 | IPI00300376 | Protein-glutamine | 77 kDa | 4.3 | 3.7 | 5.0 | 5.2 | 7.3 | 5.7 |
| | | gamma- | | | | | | | |
| | | glutamyltransferase E α | | | | | | | |
| 48 | IPI00552749 | Dynein heavy chain 8, | 478 kDa | 3.0 | 3.0 | 3.2 | 6.2 | 4.2 | 6.2 |
| | | axonemal | | | | | | | |
| 49 | IPI00396485 | Elongation factor 1-alpha | 50 kDa | 4.5 | 6.0 | 1.0 | 6.0 | 1.7 | 11.0 |
| | | 1 % | | | | | | | |
| 50 | IPI00219217 | L-lactate dehydrogenase | 37 kDa | 3.5 | 3.3 | 3.0 | 6.2 | 6.7 | 7.5 |
| | | B chain @ | | | | | | | |
| 51 | IPI00006662 | Apolipoprotein D α | 21 kDa | 1.2 | 2.7 | 2.0 | 1.2 | 6.3 | 2.3 |
| 52 | IPI00291866 | Plasma protease C1 | 55 kDa | 0.7 | 3.8 | 4.7 | 1.0 | 1.7 | 0.8 |
| | | inhibitor β | | | | | | | |
| 57 | IPI00453473 | Histone H4 % | 11 kDa | 1.3 | 5.5 | 2.7 | 4.5 | 0.5 | 9.7 |
| 58 | IPI00008290 | Ephrin type-A receptor 5 α | 115 kDa | 0.5 | 0.7 | 1.5 | 5.2 | 4.8 | 2.2 |
| 59 | IPI00903112 | Lactotransferrin δ | 77 kDa | 0.8 | 0.5 | 16.8 | 1.5 | 1.0 | 0.8 |
| 61 | IPI00383347 | PRO2194 α | 14 kDa | 0.8 | 1.5 | 2.0 | 2.3 | 3.3 | 2.3 |
| 65 | IPI00019502 | Myosin-9* # | 227 kDa | 0.5 | 0.5 | 0.5 | 1.0 | 0.5 | 15.7 |
| 66 | IPI00011692 | Involucrin # | 70 kDa | 0.5 | 0.5 | 0.5 | 4.3 | 0.5 | 12.3 |
| 67 | IPI00218343 | Tubulin alpha-1C chain # | 50 kDa | 0.5 | 0.7 | 0.5 | 1.3 | 0.5 | 12.3 |
| 68 | IPI00411765 | 14-3-3 protein sigma* % | 24 kDa | 0.5 | 4.0 | 0.5 | 5.7 | 1.3 | 5.8 |
| 69 | IPI00026256 | Filaggrin # | 435 kDa | 0.5 | 2.2 | 0.5 | 2.0 | 1.2 | 7.3 |
| 70 | IPI00019884 | Alpha-actinin-2 % | 104 kDa | 0.5 | 12.3 | 0.5 | 0.5 | 0.5 | 2.3 |
| 71 | IPI00303476 | ATP synthase subunit | 57 kDa | 0.7 | 8.7 | 0.5 | 0.5 | 0.5 | 5.3 |
| | | beta, mitochondrial % | | | | | | | |
| 72 | IPI00008964 | Ras-related protein Rab- | 22 kDa | 0.5 | 0.5 | 0.5 | 5.7 | 0.5 | 8.3 |
| | | 1B # | | | | | | | |
| 74 | IPI00291560 | Arginase-1* % | 35 kDa | 0.5 | 2.0 | 0.5 | 0.5 | 0.5 | 10.2 |
| 75 | IPI00217966 | L-lactate dehydrogenase # | 40 kDa | 0.5 | 0.7 | 0.5 | 3.3 | 0.5 | 9.3 |
| 77 | IPI00291467 | ADP/ATP translocase 3 % | 33 kDa | 1.5 | 4.0 | 0.7 | 0.5 | 1.0 | 3.8 |
| 78 | IPI00514201 | Myosin-6 γ | 224 kDa | 4.3 | 8.0 | 0.5 | 0.5 | 0.5 | 0.5 |
| 79 | IPI00025512 | Heat shock protein beta-1 % | 23 kDa | 0.5 | 1.8 | 0.5 | 2.0 | 0.5 | 4.3 |
| 80 | IPI00216984 | Calmodulin-like protein 3 @ | 17 kDa | 0.5 | 0.5 | 0.5 | 3.7 | 2.0 | 6.3 |
| 81 | IPI00013895 | Protein S100-A11 % | 12 kDa | 0.5 | 1.7 | 0.7 | 3.0 | 0.5 | 6.0 |
| 82 | IPI00011654 | Tubulin beta chain # | 50 kDa | 0.5 | 0.5 | 0.5 | 0.5 | 0.5 | 11.0 |
| 83 | IPI00032294 | Cystatin-S # | 16 kDa | 1.2 | 2.5 | 0.7 | 1.5 | 2.2 | 1.5 |
| 84 | IPI00908963 | ATP synthase subunit | 58 kDa | 0.5 | 6.7 | 0.5 | 0.5 | 0.5 | 4.7 |
| | | alpha % | | | | | | | |
| 85 | IPI00479186 | Pyruvate kinase isozymes | 58 kDa | 0.7 | 1.3 | 0.7 | 1.3 | 0.5 | 6.0 |
| | | M1/M2* # | | | | | | | |
| 86 | IPI00216952 | Lamin-A/C-(Progerin)* # | 65 kDa | 0.5 | 0.5 | 0.7 | 1.0 | 0.5 | 7.5 |
| 87 | IPI00304621 | Zinc finger protein 518B # | 120 kDa | 0.5 | 0.5 | 0.7 | 1.2 | 2.0 | 2.2 |
| 88 | IPI00218918 | Annexin A1 % | 39 kDa | 0.5 | 4.5 | 0.8 | 0.7 | 2.0 | 2.3 |
| 89 | IPI00419215 | Alpha-2-macroglobulin- | 161 kDa | 0.5 | 0.5 | 0.7 | 0.5 | 0.5 | 8.8 |
| | | like protein 1 # | | | | | | | |
| 90 | IPI00011229 | Cathepsin D # | 45 kDa | 0.5 | 1.0 | 1.0 | 1.3 | 1.3 | 5.2 |
| 91 | IPI00796333 | Fructose-bisphosphate | 45 kDa | 0.5 | 2.2 | 0.5 | 3.0 | 0.5 | 2.3 |
| | | aldolase A % | | | | | | | |
| 92 | IPI00020101 | Histone H2B % | 14 kDa | 0.5 | 1.5 | 1.0 | 0.5 | 0.5 | 7.0 |
| 93 | IPI00004573 | Polymeric immunoglobulin | 83 kDa | 0.5 | 0.5 | 6.7 | 0.5 | 1.3 | 0.5 |
| | | receptor δ | | | | | | | |
| 94 | IPI00386975 | Desmocollin-1* | 94 kDa | 1.5 | 1.0 | 1.3 | 0.5 | 1.5 | 1.7 |
| 95 | IPI00022426 | Protein AMBP | 39 kDa | 0.5 | 2.0 | 3.0 | 0.5 | 1.5 | 2.8 |
| 96 | IPI00017992 | Small proline-rich protein | 8 kDa | 2.0 | 0.7 | 0.5 | 2.2 | 1.5 | 1.0 |
| | | 2B @ | | | | | | | |
| 97 | IPI00023006 | Actin, alpha cardiac | 42 kDa | 0.5 | 2.8 | 1.3 | 1.3 | 0.5 | 3.0 |
| | | muscle 1 % | | | | | | | |
| 99 | IPI00465248 | Alpha-enolase* # | 47 kDa | 0.5 | 0.7 | 0.5 | 2.7 | 0.5 | 3.7 |
| 100 | IPI00414676 | Heat shock protein HSP | 83 kDa | 0.5 | 0.5 | 0.5 | 1.7 | 0.5 | 6.0 |
| | | 90-beta # | | | | | | | |
| 101 | IPI00296039 | Tropomyosin alpha-1 | 33 kDa | 0.5 | 5.7 | 0.5 | 0.5 | 0.5 | 1.0 |
| | | chain* γ | | | | | | | |
| 102 | IPI00909570 | Elongation factor 2 # | 63 kDa | 0.5 | 0.5 | 0.5 | 1.0 | 0.5 | 6.0 |
| 103 | IPI00013808 | Alpha-actinin-4 # | 105 kDa | 0.5 | 0.5 | 0.5 | 1.7 | 0.5 | 5.0 |
| 104 | IPI00643623 | Neutrophil gelatinase- | 23 kDa | 0.5 | 0.5 | 2.0 | 0.5 | 0.5 | 5.0 |
| | | associated lipocalin # | | | | | | | |
| 105 | IPI00305622 | Protein-glutamine | 90 kDa | 0.5 | 0.5 | 0.5 | 0.5 | 0.5 | 6.0 |
| | | gamma- | | | | | | | |
| | | glutamyltransferase K # | | | | | | | |
| 106 | IPI00216798 | Myosin regulatory light | 19 kDa | 0.5 | 5.7 | 0.5 | 0.5 | 0.5 | 0.5 |
| | | chain 2, ventricular/cardiac | | | | | | | |
| | | muscle isoform* γ | | | | | | | |
| 107 | IPI00335168 | Isoform Non-muscle of | 17 kDa | 0.5 | 2.3 | 0.5 | 0.5 | 0.5 | 2.7 |
| | | Myosin light polypeptide | | | | | | | |
| | | 6* % | | | | | | | |
| 108 | IPI00790739 | Aconitase 2, mitochondrial γ | 88 kDa | 0.5 | 6.3 | 0.5 | 0.5 | 0.5 | 0.5 |
| 109 | IPI00848226 | Guanine nucleotide- | 35 kDa | 0.5 | 0.5 | 0.5 | 0.5 | 0.5 | 6.0 |
| | | binding protein subunit | | | | | | | |
| | | beta-2-like 1 # | | | | | | | |
| 110 | IPI00003362 | 78 kDa glucose-regulated | 72 kDa | 0.5 | 0.5 | 0.5 | 1.3 | 0.5 | 4.7 |
| | | protein # | | | | | | | |
| 111 | IPI00412407 | Serpin B4 % | 42 kDa | 0.5 | 2.0 | 0.5 | 1.0 | 0.5 | 3.0 |
| 112 | IPI00877726 | Creatine Kinase type mu, | 50 kDa | 0.5 | 0.5 | 0.5 | 0.5 | 0.5 | 5.0 |
| | | mitochondrial* # | | | | | | | |
| 113 | IPI00426051 | Putative uncharacterized | 51 kDa | 0.5 | 0.5 | 1.0 | 1.7 | 0.5 | 2.7 |
| | | protein | | | | | | | |
| | | DKFZp686C15213 # | | | | | | | |
| 114 | IPI00419585 | Peptidyl-prolyl cis-trans | 18 kDa | 0.5 | 0.7 | 0.5 | 1.3 | 0.5 | 3.3 |
| | | isomerase A # | | | | | | | |
| 115 | IPI00893099 | Heat shock 70 kDa protein | 70 kDa | 0.5 | 0.5 | 0.5 | 1.7 | 0.5 | 3.3 |
| | | 1-like variant # | | | | | | | |
| 116 | IPI00794543 | Calmodulin # | 17 kDa | 0.5 | 0.5 | 0.5 | 1.0 | 0.5 | 4.3 |
| 117 | IPI00219757 | Glutathione S-transferase | 23 kDa | 0.5 | 0.5 | 0.5 | 1.7 | 0.5 | 3.3 |
| | | P # | | | | | | | |
| 118 | IPI00021828 | Cystatin-B % | 11 kDa | 0.5 | 1.7 | 0.5 | 0.5 | 0.5 | 3.0 |
| 119 | IPI00291922 | Proteasome subunit alpha | 26 kDa | 0.5 | 0.5 | 0.5 | 0.5 | 0.5 | 4.0 |
| | | type-5 # | | | | | | | |
| 120 | IPI00021812 | Neuroblast | 629 kDa | 0.5 | 0.5 | 0.5 | 0.5 | 0.5 | 3.3 |
| | | differentiation-associated | | | | | | | |
| | | protein # | | | | | | | |
| 121 | IPI00060800 | Zymogen granule protein | 23 kDa | 0.5 | 0.5 | 2.0 | 2.0 | 0.5 | 0.5 |
| | | 16 homolog B δ | | | | | | | |
| 123 | IPI00291006 | Malate dehydrogenase, | 36 kDa | 0.5 | 2.0 | 0.5 | 0.5 | 0.5 | 1.0 |
| | | mitochondrial % | | | | | | | |
| 124 | IPI00215917 | ADP-ribosylation factor 3 # | 21 kDa | 0.7 | 0.5 | 0.5 | 0.5 | 0.5 | 3.7 |
| 125 | IPI00009856 | Protein Plunc | 27 kDa | 0.5 | 0.5 | 3.7 | 0.5 | 0.5 | 0.7 |
| 126 | IPI00215965 | Heterogeneous nuclear | 39 kDa | 0.5 | 0.5 | 0.5 | 0.5 | 0.5 | 3.3 |
| | | ribonucleoprotein A1* # | | | | | | | |
| 127 | IPI00216026 | Voltage-dependent anion- | 32 kDa | 0.5 | 0.5 | 0.5 | 0.5 | 0.5 | 3.3 |
| | | selective channel protein | | | | | | | |
| | | 2* # | | | | | | | |
| 128 | IPI00873099 | Protein S100A2 # | 11 kDa | 0.5 | 0.5 | 0.5 | 0.5 | 0.5 | 2.3 |
| 129 | IPI00414684 | Semenogelin-1* δ | 45 kDa | 0.5 | 0.5 | 3.7 | 0.5 | 0.5 | 0.5 |
| 130 | IPI00797270 | Triosephosphate | 27 kDa | 0.5 | 1.5 | 0.5 | 1.7 | 0.5 | 1.3 |
| | | isomerase % | | | | | | | |
| 132 | IPI00022774 | Transitional endoplasmic | 89 kDa | 0.5 | 0.5 | 0.5 | 0.7 | 0.5 | 2.3 |
| | | reticulum ATPase # | | | | | | | |
| 134 | IPI00879238 | 40S ribosomal protein S9 # | 17 kDa | 0.5 | 0.5 | 0.5 | 0.5 | 0.5 | 2.3 |
| 136 | IPI00216975 | Tropomyosin alpha-4 | 33 kDa | 0.5 | 0.5 | 0.7 | 0.5 | 0.5 | 2.0 |
| | | chain* # | | | | | | | |
| 137 | IPI00232492 | Tripartite motif- | 64 kDa | 0.5 | 0.5 | 0.5 | 0.5 | 0.5 | 3.0 |
| | | containing protein 29* # | | | | | | | |
| 138 | IPI00017672 | Purine nucleoside | 33 kDa | 0.5 | 0.5 | 0.5 | 0.5 | 0.5 | 3.0 |
| | | phosphorylase # | | | | | | | |
| 139 | IPI00007188 | ADP/ATP translocase 2 # | 33 kDa | 0.5 | 0.5 | 0.5 | 0.5 | 0.5 | 3.0 |
| 140 | IPI00243742 | Myosin light chain 3 γ | 22 kDa | 0.5 | 2.7 | 0.5 | 0.5 | 0.5 | 0.5 |
| 141 | IPI00291410 | Long palate, lung and nasal | 52 kDa | 0.5 | 0.5 | 1.7 | 1.0 | 0.7 | 0.5 |
| | | epithelium carcinoma- | | | | | | | |
| | | associated protein 1* δ | | | | | | | |
| 143 | IPI00216691 | Profilin-1δ | 15 kDa | 0.5 | 0.5 | 2.3 | 0.7 | 0.5 | 0.5 |
| 144 | IPI00790304 | Voltage-dependent anion- | 20 kDa | 0.5 | 0.5 | 0.5 | 0.5 | 0.5 | 2.7 |
| | | selective channel protein 1 # | | | | | | | |
| 145 | IPI00015141 | Creatine kinase, sarcomeric | 48 kDa | 0.5 | 2.7 | 0.5 | 0.5 | 0.5 | 0.5 |
| | | mitochondrial γ | | | | | | | |
| 146 | IPI00244346 | Troponin I, cardiac muscle γ | 24 kDa | 0.5 | 2.7 | 0.5 | 0.5 | 0.5 | 0.5 |
| 147 | IPI00556485 | 60S acidic ribosomal | 27 kDa | 0.5 | 0.5 | 0.5 | 0.5 | 0.5 | 2.7 |
| | | protein P0 # | | | | | | | |
| 148 | IPI00012011 | Cofilin-1 # | 19 kDa | 0.5 | 0.5 | 0.5 | 0.7 | 0.5 | 2.0 |
| 149 | IPI00915941 | 60 kDa heat shock | 25 kDa | 0.5 | 0.7 | 0.5 | 0.5 | 0.5 | 1.7 |
| | | protein, mitochondrial # | | | | | | | |
| 150 | IPI00186711 | Plectin-1* # | 518 kDa | 0.5 | 0.5 | 0.7 | 0.5 | 0.5 | 1.7 |
| 151 | IPI00455383 | Clathrin heavy chain 1* # | 188 kDa | 0.5 | 0.5 | 0.5 | 0.5 | 0.5 | 2.0 |
| 152 | IPI00328328 | Eukaryotic initiation | 46 kDa | 0.5 | 0.5 | 0.5 | 0.5 | 0.5 | 2.3 |
| | | factor 4A-II* # | | | | | | | |
| 153 | IPI00479306 | Proteasome subunit beta | 28 kDa | 0.5 | 0.5 | 0.5 | 0.5 | 0.5 | 2.3 |
| | | type-5 # | | | | | | | |
| 154 | IPI00926685 | Tubulin beta-4 chain # | 41 kDa | 0.5 | 0.5 | 0.5 | 0.5 | 0.5 | 2.0 |
| 155 | IPI00010214 | Protein S100-A14 # | 12 kDa | 0.5 | 0.5 | 0.5 | 0.5 | 0.5 | 2.3 |
| 156 | IPI00382482 | Ig heavy chain V-III | 14 kDa | 0.5 | 0.5 | 0.5 | 0.5 | 0.8 | 1.2 |
| | | region CAM # | | | | | | | |
| 157 | IPI00219575 | Bleomycin hydrolase # | 53 kDa | 0.5 | 0.5 | 0.5 | 0.5 | 0.5 | 1.7 |
| 158 | IPI00798035 | Myosin-binding protein C, | 141 kDa | 0.5 | 2.3 | 0.5 | 0.5 | 0.5 | 0.5 |
| | | cardiac-type γ | | | | | | | |
| 159 | IPI00025491 | Eukaryotic initiation | 46 kDa | 0.5 | 0.5 | 0.5 | 0.5 | 0.5 | 1.7 |
| | | factor 4A-I # | | | | | | | |
| 160 | IPI00329389 | 60S ribosomal protein L6# | 33 kDa | 0.5 | 0.5 | 0.5 | 0.5 | 0.5 | 1.7 |
| 161 | IPI00645201 | Ribosomal protein S8 # | 22 kDa | 0.5 | 0.5 | 0.5 | 0.5 | 0.5 | 1.7 |
| 162 | IPI00289983 | Prostatic acid | 48 kDa | 0.5 | 0.5 | 0.5 | 0.5 | 0.5 | 1.3 |
| | | phosphatase* # | | | | | | | |
| 163 | IPI00925023 | NADH-ubiquinone | 74 kDa | 0.5 | 2.0 | 0.5 | 0.5 | 0.5 | 0.5 |
| | | oxidoreductase 75 kDa | | | | | | | |
| | | subunit, mitochondrial γ | | | | | | | |
| 164 | IPI00473011 | Hemoglobin subunit delta # | 16 kDa | 0.5 | 0.7 | 0.5 | 0.5 | 0.5 | 1.7 |
| 165 | IPI00018146 | 14-3-3 protein theta # | 28 kDa | 0.5 | 0.5 | 0.5 | 0.5 | 0.5 | 2.0 |
| 166 | IPI00154509 | Proteasome subunit alpha | 29 kDa | 0.5 | 0.5 | 0.5 | 0.5 | 0.5 | 2.0 |
| | | type-7-like # | | | | | | | |
| 167 | IPI00759776 | Actinin, alpha 1* i # | 106 kDa | 0.5 | 0.5 | 0.5 | 0.5 | 0.5 | 2.0 |
| 168 | IPI00909534 | Elongation factor 1- | 24 kDa | 0.5 | 0.5 | 0.5 | 0.5 | 0.5 | 2.0 |
| | | gamma # | | | | | | | |
| 169 | IPI00414696 | Heterogeneous nuclear | 36 kDa | 0.5 | 0.5 | 0.5 | 0.5 | 0.5 | 1.8 |
| | | ribonucleoproteins | | | | | | | |
| | | A2/B1* # | | | | | | | |
| 172 | IPI00216318 | 14-3-3 protein beta/alpha* # | 28 kDa | 0.5 | 0.5 | 0.5 | 0.5 | 0.5 | 1.3 |
| 173 | IPI00550363 | Transgelin-2 # | 22 kDa | 0.5 | 0.5 | 0.5 | 0.5 | 0.5 | 1.7 |
| 174 | IPI00301021 | Translocon-associated | 32 kDa | 0.5 | 0.5 | 0.5 | 0.5 | 0.5 | 1.7 |
| | | protein subunit alpha* # | | | | | | | |
| 175 | IPI00871956 | Similar to 40S ribosomal | 20 kDa | 0.5 | 0.5 | 0.5 | 0.5 | 0.5 | 1.7 |
| | | protein S2 # | | | | | | | |
| 177 | IPI00220740 | Nucleophosmin* # | 29 kDa | 0.5 | 0.5 | 0.5 | 0.5 | 0.5 | 1.7 |
| 178 | IPI00023635 | Inositol monophosphatase | 31 kDa | 0.5 | 0.5 | 0.5 | 0.5 | 0.5 | 1.3 |
| | | 2* # | | | | | | | |
| 179 | IPI00031549 | Desmocollin-3* # | 100 kDa | 0.5 | 0.5 | 0.5 | 0.5 | 0.5 | 1.0 |
| 180 | IPI00555956 | Proteasome subunit beta | 29 kDa | 0.5 | 0.5 | 0.5 | 0.5 | 0.5 | 1.0 |
| | | type-4 # | | | | | | | |
| 181 | IPI00478287 | Putative uncharacterized | 22 kDa | 0.5 | 0.5 | 0.5 | 0.5 | 0.5 | 1.3 |
| | | protein | | | | | | | |
| | | ENSP00000352132 # | | | | | | | |
| 182 | IPI00219038 | Histone H3.3 # | 15 kDa | 0.5 | 0.5 | 0.5 | 0.5 | 0.5 | 1.0 |
| 184 | IPI00941747 | Calnexin # | 68 kDa | 0.5 | 0.5 | 0.5 | 0.5 | 0.5 | 1.0 |
| 185 | IPI00219622 | Proteasome subunit alpha | 26 kDa | 0.5 | 0.5 | 0.5 | 0.5 | 0.5 | 1.0 |
| | | type-2 # | | | | | | | |
| 186 | IPI00873506 | Guanine aminohydrolase # | 53 kDa | 0.5 | 0.5 | 0.5 | 0.5 | 0.5 | 1.0 |

Footnote
All isoforms are covered for proteins marked with an asterisk (*)
TP1—Baseline before surgery
TP7—1 hr post PTCA
TP8—24 hr Post PTCA
Proteins in bold are elevated in diseased group at either TP7 or TP8
Proteins in italics are decreased in diseased group based at either TP7 or TP8
# elevated by at least two fold in diseased at TP8 only
@ elevated by at least two fold at in diseased at TP7 and remain elevated at TP8
α—Elevated by at least two fold in diseased at TP7 and return to baseline at TP8
% decreased by at least two fold in diseased at TP7 and increase by at least two fold in diseased at TP8
β—decreased by at least two fold in diseased at TP7 and remain decreased at TP8
γ—decreased by at least two fold in diseased at TP7 and return to baseline at TP8
δ—decreased by at least two fold in diseased at TP8 only

The method disclosed herein can be used alone, or in conjunction with other diagnostic tests to improve the accuracy and specificity of the diagnosis. These include commonally used myocardial injury biomarkers like cTnI, cTnT, myoglobin, CKMB. The method can also be used for screening purposes, to identify individuals who appear to be “at risk” for further testing by this or other means.

Accordingly, in one aspect, the method comprises (a) determining the level of at least one biomarker in a biological sample obtained from said subject, wherein said biomarker comprises a protein or peptide identified in Table 2, and (b) an elevation or decrease in the level of the biomarker, compared to control level of certain proteins or peptides, is indicative of a disease or disorder. In an embodiment, the disease is ischemia. In another embodiment, the disease is myocardial ischemia. In another embodiment, the disease is renal ischemia. In another embodiment, the disease is skeletal muscle ischemia. In another embodiment, the disease is brain ischemia. In another embodiment, the disease is organ ischemia.

In another aspect, the method comprises assaying a subject sample for the presence of at least one biomarker comprising a protein/peptide of Table 2; wherein the detection of said biomarker(s) is correlated with a diagnosis of the disease or disorder, the correlation taking into account the presence and level of biomarker(s) in the subject sample as compared to normal subjects.

The biomarkers can be detected by any suitable means known to those of skill in the art, for example, using a protein or peptide assay, binding assay, or an immunoassay. Biomarkers may also be identified as peaks using Mass Spectroscopy (MS) of the intact or digested peptide(s), or as gel bands using, for example size exclusion chromatography (SEC), optionally after appropriate initial treatment of the sample after isolation of ABBPC. For a positive diagnosis, the biomarkers are elevated or lowered as compared to values in normal healthy controls or changes in the same individual over time can be used. Multiple reaction monitoring (MRM) is a mass spectrometry technique that allows monitoring of selected ions which is useful in another embodiment. Using this technique one can monitor very specific chemical or biological species and can obtain absolute quantitation. For example, you can determine the concentration of a protein based on the monitoring of one or more peptides unique to that protein.

The subject sample may be selected, for example, from the group consisting of blood, blood plasma, serum or other body fluids. Preferably, the sample is albumin-enriched serum or plasma.

The diagnostic assay can be used, for example, to evaluate patients presenting to an emergency room, or for ongoing care within a hospital setting, or in a medical practitioner's office or in emergency transit (eg ambulance), during or following surgery or therapeutic treatment (e.g. during or following angioplasty or thrombylsis treatment). The assay has the advantage that it can be easily and reproducibly obtained from individuals since albumin is highly abundant in serum (40-50 mg/ml). Specific antibodies to albumin are available and the ABPPC can be enriched or captured easily without a complicated assay. Other biochemical methods can be used as well, including liquid chromatography, affinity chromatography, and gel based methods. Capturing this naturally-occurring sub-proteome reduces sample complexity and avoids the problems associated with assay sensitivity at low protein concentrations. Since some proteins in the ABPPC have not been observed in albumin depleted serum, it appears that some biomarkers are unique to the ABPPC.

Also provided is a kit for carrying out the method described herein. In one embodiment, the kit may comprise any of: an antibody (or a chemical moiety) to specifically capture or enrich for the endogenous albumin, a secondary antibody (or chemical moiety) to one or more of the specific protein (or peptide or modified protein) bound to albumin and components for detection and/or quantification of the amount of secondary antibody bound. In one embodiment, the secondary antibody would be against protein(s) listed in Table 1 or Table 2 that change in ischemia with the specific protein so that one is quantifying the change in protein content of the ABPPC.

In an embodiment, endogenous ABPPC is captured (with an antibody or chemical moiety) followed by a direct detection of the protein(s) of interest using mass spectrometry (MS) of the intact or enzymatically degraded protein. In this embodiment the kit may contain the anti-albumin antibody coupled to a matrix (for example, in a small column or packed into an end of a pipette tip) where the ABPPC would be enriched following elution into MS for intact mass or eluted for digestion and subsequent MS analysis (of all peptides or specific signature peptide for the analyte(s)). The kit may further comprise a labeled internal protein standard. Kits of the invention may contain a plurality of antibodies so that more than one ABPPC component could be assessed simultaneously.

It is also believed that the ratio of bound to free (circulating) ABPPC may be important. Methods and kits may be modified so that specific proteins are measured as bound to serum albumin or free. For example, a number of proteins have been observed to be both bound to albumin, but also observed in the albumin-depleted fraction of serum, indicating that they could be present in their free form. Examples of these proteins include antithrombin III, apolipoprotein AIL AIV, CII, clusterin, transthyretin, and vitamin D binding protein, for example. Practitioners will be able to determine through routine experimentation how the ratio is altered in particular disease states.

Diseases or disorders for which the methods and compositions of the invention are expected to be useful include ischemia. Different forms of ischemia may be detectable including myocardial ischemia, organ ischemia, renal ischemia, and brain ischemia.

### DEFINITIONS

The following terms are used as defined below throughout this application, unless otherwise indicated.

“Marker” or “biomarker” are used interchangeably herein, and in the context of the present invention refer to an ABPPC (of a particular specific identity or apparent molecular weight) which is differentially present in a sample taken from patients having a specific disease or disorder as compared to a control value, the control value consisting of, for example, average or mean values in comparable samples taken from control subjects (e.g., a person with a negative diagnosis, normal or healthy subject). Biomarkers may be determined as specific peptides or proteins (Table 1 or Table 2), either presently bound or cleaved from albumin, or as specific peaks, bands, fractions, etc. in a mass spectroscopy, size exclusion chromatography, or other separation process or antibody detection. In some applications, for example, a mass spectroscopy or other profile or multiple antibodies may be used to determine multiple biomarkers, and differences between individual biomarkers and/or the partial or complete profile may be used for diagnosis.

The phrase “differentially present” refers to differences in the quantity and/or the frequency of a marker present in a sample taken from patients having a specific disease or disorder as compared to a control subject. For example, a marker can be a ABPPC which is present at an elevated level or at a decreased level in samples of patients with the disease or disorder compared to a control value (e.g. determined from samples of control subjects). Alternatively, a marker can be an ABPPC which is detected at a higher frequency or at a lower frequency in samples of patients compared to samples of control subjects. A marker can be differentially present in terms of quantity, frequency or both. It may also be a physical change/modification of the protein that is the marker, rather than just an increase or decrease in the amount present/detected. For example, it may be the post-translational modification, cleavage, or isoform of the protein that is changing, and it is this change that is detected by the assay. This is separate from determining a different quantity in diseased vs. control.

A marker, compound, composition or substance is differentially present in a sample if the amount of the marker, compound, composition or substance in the sample is statistically significantly different from the amount of the marker, compound, composition or substance in another sample, or from a control value. For example, a compound is differentially present if it is present at least about 120%, at least about 130%, at least about 150%, at least about 180%, at least about 200%, at least about 300%, at least about 500%, at least about 700%, at least about 900%, or at least about 1000% greater or less than it is present in the other sample (e.g. control), or if it is detectable in one sample and not detectable in the other.

Alternatively or additionally, a marker, compound, composition or substance is differentially present between samples if the frequency of detecting the marker, etc. in samples of patients suffering from a particular disease or disorder, is statistically significantly higher or lower than in the control samples or control values obtained from healthy individuals. For example, a biomarker is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples. These exemplary values notwithstanding, it is expected that a skilled practitioner can determine cut-off points, etc. that represent a statistically significant difference to determine whether the marker is differentially present.

“Diagnostic” means identifying the presence or nature of a pathologic condition and includes identifying patients who are at risk of developing a specific disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “detection”, “detecting” and the like, may be used in the context of detecting biomarkers, or of detecting a disease or disorder (e.g. when positive assay results are obtained). In the latter context, “detecting” and “diagnosing” are considered synonymous.

By “at risk of” is intended to mean at increased risk of, compared to a normal subject, or compared to a control group, e.g. a patient population. Thus a subject carrying a particular marker may have an increased risk for a specific disease or disorder, and be identified as needing further testing. “Increased risk” or “elevated risk” mean any statistically significant increase in the probability, e.g., that the subject has the disorder. The risk is preferably increased by at least 10%, more preferably at least 20%, and even more preferably at least 50% over the control group with which the comparison is being made.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of a particular disease or disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder sought to be diagnosed. A control amount can be either in absolute amount (e.g., μg/ml) or a relative amount (e.g., relative intensity of signals).

The terms “polypeptide,” “peptide” and “protein” are used interchangeably herein to refer to a polymer of α-amino acid residues, in particular, of naturally-occurring α-amino acids. The terms apply to amino acid polymers in which one or more amino acid residue is an analog or mimetic of a corresponding naturally-occurring amino acid, as well as to naturally-occurring amino acid polymers. Polypeptides can be modified, e.g., by the addition of carbohydrate residues to form glycoproteins, phosphorylation to form phosphoproteins, and a large number of chemical modifications (oxidation, deamidation, amidation, methylation, formylation, hydroxymethylation, guanidination, for example) as well as degraded, reduced, or crosslinked. The terms “polypeptide,” “peptide” and “protein” include all unmodified and modified forms of the protein.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact or subsequentally digested peptides (one or more peptide can be assessed.)

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

By “binding assay” is meant a biochemical assay wherein the biomarkers are detected by binding to an agent, such as an antibody, through which the detection process is carried out. The detection process may involve radioactive or fluorescent labels, and the like. The assay may involve immobilization of the biomarker, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The terms “subject”, “patient” or “individual” generally refer to a human, although the methods of the invention are not limited to humans, and should be useful in other animals (e.g. birds, reptiles, amphibians, mammals), particularly in mammals, since albumin is homologous among species.

“Sample” is used herein in its broadest sense. A sample may comprise a bodily fluid including blood, serum, plasma, tears, aqueous and vitreous humor, spinal fluid; a soluble fraction of a cell or tissue preparation, or media in which cells were grown; a, aorganelle, or membrane isolated or extracted from a cell or tissue; polypeptides, or peptides in solution or bound to a substrate; a cell; a tissue; a tissue print; a fingerprint, skin or hair; fragments and derivatives thereof. Subject samples usually comprise derivatives of blood products, including blood, plasma and serum.

By “albumin-enriched serum or plasma” is meant serum or plasma that has been treated to reduce or remove components other than albumin and associated peptides and proteins which are bound thereto.

### EXAMPLES

There are two primary methods available for isolating albumin from serum or plasma: affinity-based (e.g., antibody, Cibacron blue) and chemical-based methods (e.g., NaCl/EtOH (Fu, Q., Garnham, C. P., Elliott, S. T., Bovenkamp, D. E. et al., Proteomics 2005, 5, 2656-2664. Colantonio, D. A., Dunkinson, C., Bovenkamp, D. E., Van Eyk, J. E., Proteomics 2005, 5, 3831-3835.) TCA/acetone (Chen, Y. Y., Lin, S. Y., Yeh, Y. Y., Hsiao, H. H. et al., Electrophoresis 2005, 26, 2117-2127)). Many of the affinity-based methods have been compared and shown to effectively remove albumin (Zolotarjova, N., Martosella, J., Nicol, G., Bailey, J. et al., Proteomics 2005, 5, 3304-3313; Björhall, K., Miliotis, T., Davidsson, P., Proteomics 2005, 5, 307-317; Chromy, B. A., Gonzales, A. D., Perkins, J., Choi, M. W. et al., J. Proteome Res. 2004, 3, 1120-1127). However, these methods are vulnerable to non-specific binding of proteins/peptides to the ligand and column materials and carryover between experiments in the case of LC columns (Zolotarjova, N., Martosella, J., Nicol, G., Bailey, J. et al., Proteomics 2005, 5, 3304-3313; Colantonio, D. A., Dunkinson, C., Bovenkamp, D. E., Van Eyk, J. E., Proteomics 2005, 5, 3831-3835; Björhall, K., Miliotis, T., Davidsson, P., Proteomics 2005, 5, 307-317; Chromy, B. A., Gonzales, A. D., Perkins, J., Choi, M. W. et al., J. Proteome Res. 2004, 3, 1120-1127; Steel, L. F., Trotter, M. G., Nakajima, P. B., Mattu, T. S. et al., Mol. Cell. Proteomics 2003, 2, 262-270; Stanley, B. A., Gundry, R. L., Cotter, R. J., Van Eyk, J. E., Dis. Markers 2004, 20, 167-178). Alternatively, albumin has been purified using NaCl/EtOH precipitation since the 1940s (Cohn, E. J., Strong, L. E., Hughes, W. L., Mulford, D. J. et al., J. Am. Chem. Soc. 1946, 68, 459-475) and this method is routinely used for isolating pharmaceutical grade albumin. Recently, this process was optimized for the proteomics field to minimize the steps required for effective purification and removal of albumin (Fu, Q., Garnham, C. P., Elliott, S. T., Bovenkamp, D. E. et al., Proteomics 2005, 5, 2656-2664), but copurification of other proteins may still be an issue.

### Example 1

Cohort:

Human serum was obtained from patients undergoing elective angioplasty (PTCA). Serum was drawn from the femoral artery at various time points throughout the procedure. The patient samples were classified as non-diseased (control) or diseased (myocardial infarction, MI) based on the absence or presence of cardiac troponin I (cTnI), respectively. Three time points from each group were chosen for analysis, T₀—baseline, T₇—1 r post PTCA, and T₈—24 hr post PTCA.

Materials:

All reagents and solvents were of the highest grade available. Size exclusion standards were all purchased from Sigma Aldrich and were at least 90% pure.

Size Exclusion Chromatography:

Human serum albumin (HSA) was removed from the serum samples by chemical depletion, in which non-HSA associated proteins are precipitated using a NaCl/EtOH solvent system and HSA and its associated proteins/peptides remain in the supernatant. The HSA containing supernatant was then subjected to non-denaturing size exclusion chromatography (SEC) performed on a ProteomeLab PF2D HPLC system (Beckman Coulter, Fullerton, Calif., USA) using a BioSep-SEC-S2000 300×7.8 mm column (Phenomenex, Torrance, Calif., USA). The mobile phase was 50 mM sodium phosphate buffer, pH 6.8, which was run isocratically at a flow rate of 0.25 mL/min. For each sample, 200 μg of total protein was loaded onto the SEC column two times and fractions from both runs were combined. Fractions were collected every 0.5 minutes and fractions that contained HSA with associated proteins/peptides bound were collected and pooled together in 2-minute fraction pools over 10 minutes (fractions labeled A→E). Fractions A and B were then combined to give fraction AB, so there were four total pooled SEC fractions for each sample. Total protein concentration for each pooled fraction (AB, C, D, and E) was determined using a micro BCA assay kit (Sigma Aldrich, St. Louis, Mo., USA) according to the manufacturer's protocol. Six molecular weight standards were also run using the same experimental conditions (Beta-galactosidase from Aspergillus oryzae 116.3 kDa, human serum albumin 67 kDa, chicken ovalbumin 45 kDa, carbonic anhydrase from bovine erythrocytes 30 kDa, myoglobin from equine heart 16.7 kDa, and bovine oxidized insulin beta-chain 3.5 kDa).

1-D SDS-PAGE and Tryptic Digestion:

Three hundred and seventy five nanograms of total protein from each fraction pool was then lyophilized and protein was resuspended in a 3:1 mixture of 20 mM DTT:4× Invitrogen Loading buffer. Samples were then boiled at 95° C. for 5 min and loaded onto Invitrogen 4-12% Bis-Tris gels. Gels were run in 1×MES running buffer at 140V for 20 min then at 200V until tracking dye reached the bottom of the gel. Gels were silver stained according to the protocol of Shevchenko et al. (Shevchenko et al. Analytical Chemistry 1996, 68:850-858). The bands corresponding to albumin and the albumin dimer were excised from the gels and discarded. The remaining gel from each lane was then placed in a 2.0 mL eppendorf tube and digested with trypsin.

Mass Spectrometry:

Peptide solutions for each pooled fraction were desalted using Omix C18 ZipTips (Varian, Santa Clara, Calif., USA) according to the manufacturer's protocol and eluted with 30 μL of 70% acetonitrile (MeCN), 0.1% formic acid (FA). Two microliters of fractions AB and C were combined and 2 μL of fractions D and E were combined before LC-MS/MS analysis. Two technical replicates of each combination were analyzed on an Agilent 1200 nano-LC system (Agilent, Santa Clara, Calif., USA) connected to an LTQ-Orbitrap mass spectrometer (Thermo, Waltham, Mass., USA) equipped with a nanoelectrospray ion source. Peptides were separated on a C₁₈ RP-HPLC column (75 μm×10 cm self-packed with 5 μm, 200 Å Magic C18; Michrom BioResources, Auburn, Calif., USA) at a flow rate of 300 nl/min where mobile phase A was 0.1% v/v formic acid in water and mobile phase B was 90% acetonitrile, 0.1% formic acid in water. The linear gradient was 10-45% B in 40 minutes. Each MS1 scan followed by collision induced dissociation (CID, acquired in the LTQ part) of the seven most abundant precursor ions with dynamic exclusion for 24 seconds. Only MS1 signals exceeding 1000 counts triggered the MS2 scans. For MS1, 2×10⁵ ions were accumulated in the Orbitrap over a maximum time of 500 ms and scanned at a resolution of 60,000 FWHM (from 375-2000 m/z). MS2 spectra (via collision induced dissociation (CID)) were acquired in normal scan mode in the LTQ, with a target setting of 10⁴ ions and accumulation time of 30 ms. The normalized collision energy was set to 35%, and one microscan was acquired for each spectrum. An exclusion list of 134 m/z values corresponding to human serum albumin and bovine pancreatic trypsin peptides was generated based on previous MS runs, which excluded these values from being selected for MS2 analysis.

Database Searching.

Raw MS data were searched against the International Protein Index human v.3.62 database was performed using Sorcerer 2™-SEQUEST® (Sage-N Research, Milpitas, Calif., USA) with post-search analysis performed using Scaffold 3 (Proteome Software, Inc., Portland, Oreg., USA). All raw data peak extraction was performed using Sorcerer 2-SEQUEST default settings. Database search parameters were as follows: semi-enzyme digest using trypsin (after Lys or Arg) with up to 2 missed cleavages; monoisotopic precursor mass range of 400-4500 amu; differential oxidation of methionine and static carbamidomethylation of cysteine were allowed. Peptide mass tolerance was set to 50 ppm, fragment mass type was set to monoisotopic, and maximum number of modifications set to 4 per peptide. Advanced search options that were enabled included: XCorr score cutoff of 1.5; isotope check using mass shift of 1.003355 amu; keep the top2000 preliminary results for final scoring; display up to 200 peptide results in the result file; display up to 5 full protein descriptions in the result file; display up to 1 duplicate protein references in the result file. Error rates (false discovery rates) and protein probabilities (p) were calculated by Scaffold. The raw data from each AB-C and D-E duplicate for each sample were combined into a single database search.

### Results

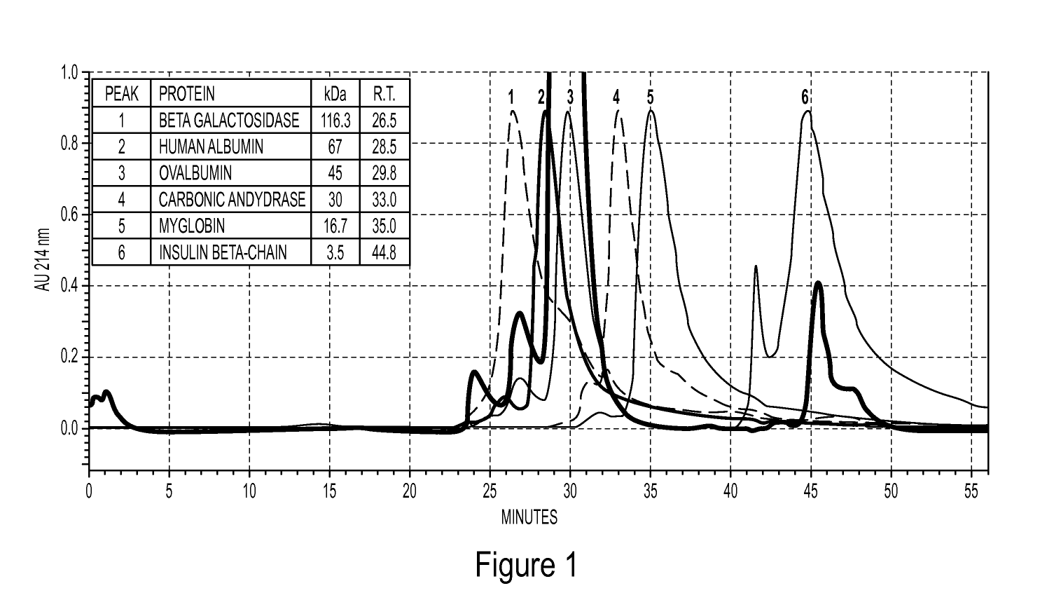

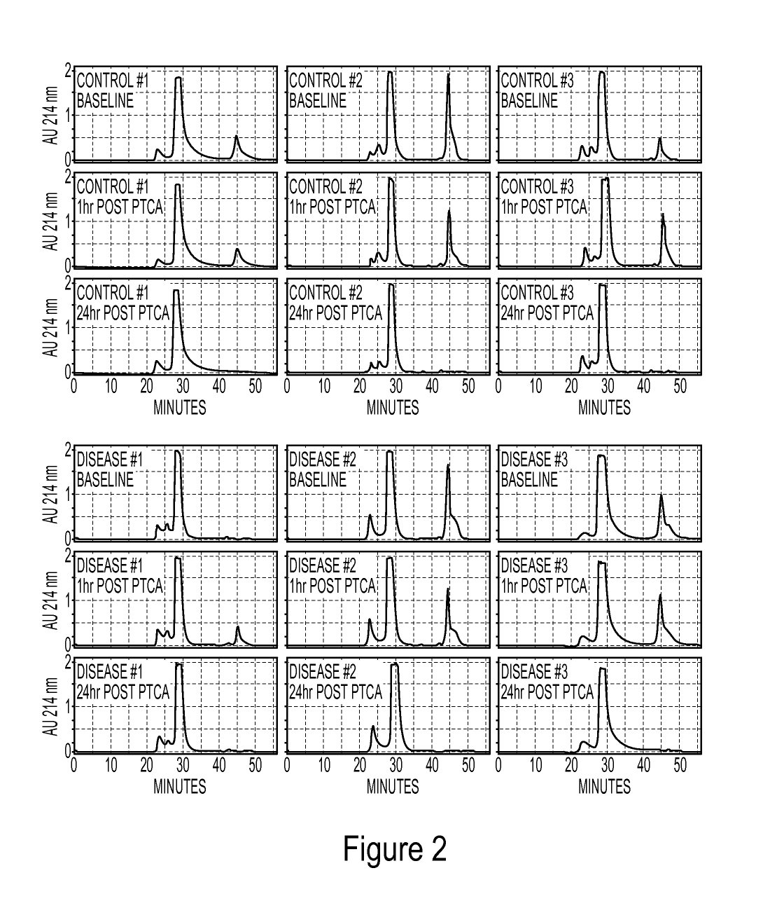

The serum of six patients (3 control, 3 diseased) undergoing elective angioplasty (PTCA) was collected at three time-points as described above. The ABPPC from each of these samples was analyzed by size exclusion chromatography (SEC), 1-D electrophoresis and LC-MS/MS. Molecular weight standards were run on the SEC column before analysis of the ABPPC samples and the chromatogram is shown in FIG. 1. Size exclusion chromatograms for each ABPPC sample are shown in FIG. 2.

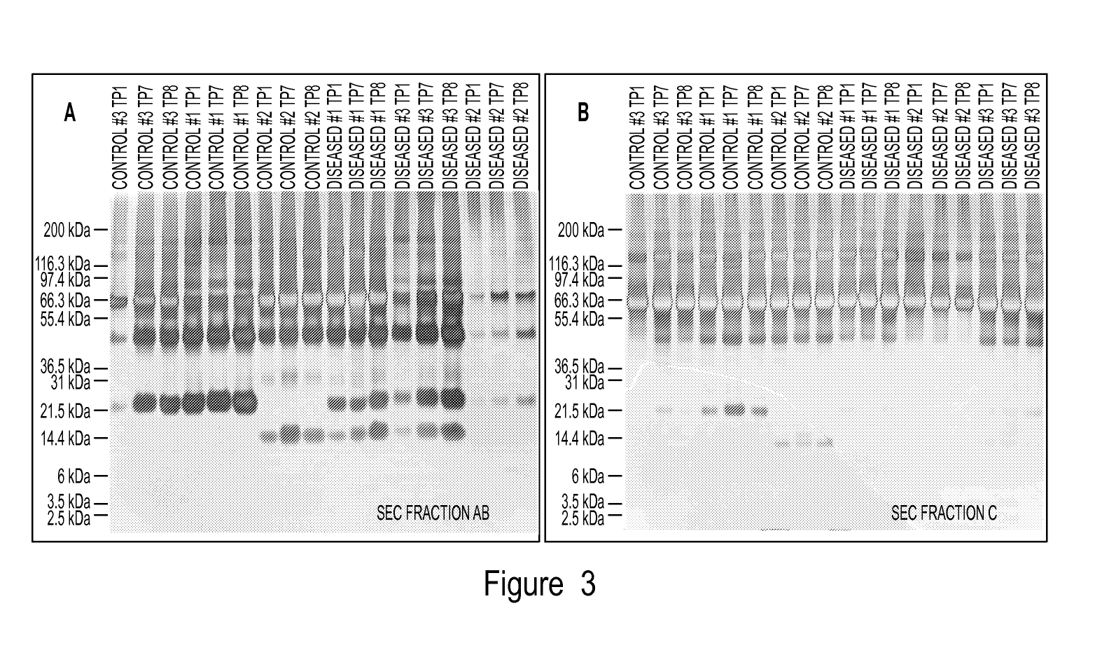

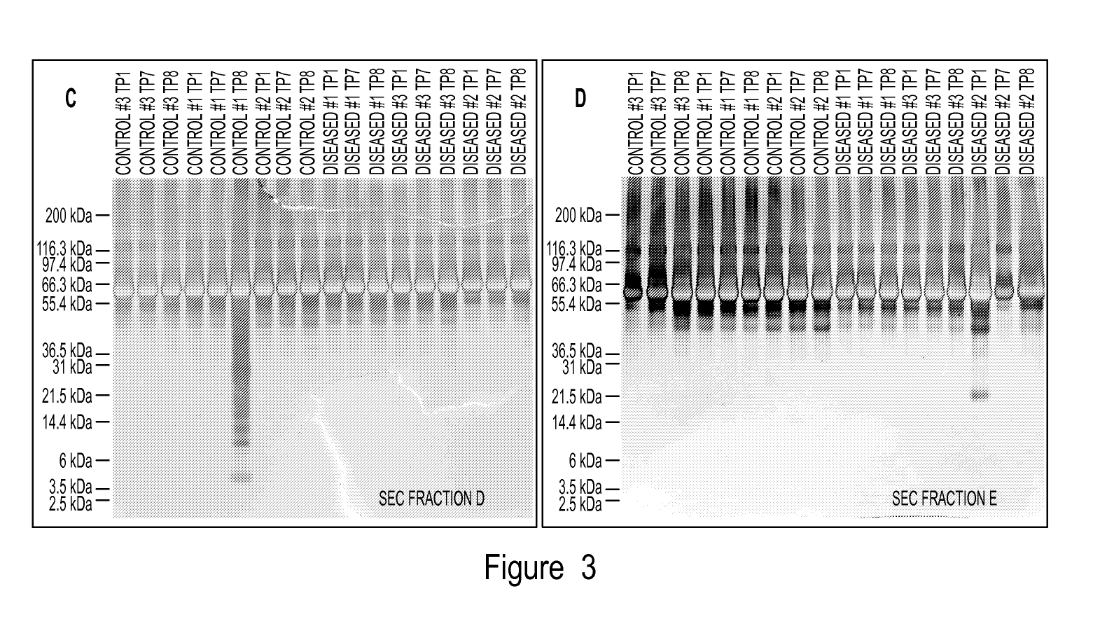

Looking at the SEC chromatograms in FIG. 2 it is clear that the ABPPC is indeed different between individuals, which show that there is biological variation in the ABPPC between patients. Additionally, the ABPPC is also changing within patients, as can be seen by the change in the small peaks between 22 and 28 minutes for some patients. The 1-DE gel profiles for the SEC fractions are also different between patients (FIG. 3), especially in the AB and C fractions, which were collected between 22.5-26 and 26.5-28 minutes, respectively. These fractions correspond to the small peaks that are eluting in the MW range of greater than 66 kDa, as shown in FIG. 1. This region of the SEC is most likely where the majority of the ABPPC is located so this is further evidence that there is biological variation between individuals.

The large peak between 45-50 minutes (corresponding to a MW of about 3,500 Da, determined from the chromatogram of MW standards) seen in some of the SEC chromatograms has yet to be identified. The fractions ranging from 44-50 minutes were collected and, although these pooled fractions reported an absorbance at 595 nm when assayed by BCA method, the 1-DE SDS-PAGE did not show any bands for this fraction when they were silver stained (results not shown). In addition, trypsin digestion and LC-MS/MS analysis of these fractions did not show the presence of any human protein or peptide.

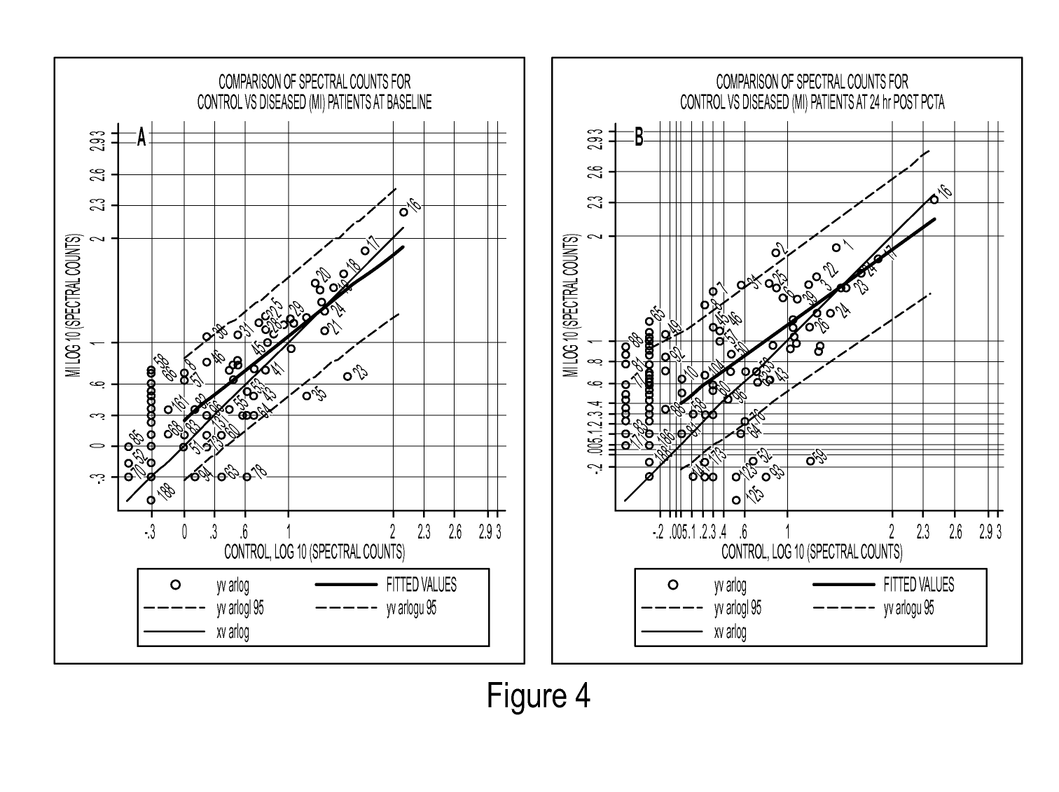

The gel pieces (minus albumin) for each fraction were digested with trypsin and analyzed by LC-MS/MS. A search of the human IPI database returned 187 total proteins that were distributed throughout the samples. A majority of these proteins were present only in the disease #2, 24 hr post PTCA sample. Proteins reporting a zero spectral count were arbitrarily assigned a value of 0.5. For data analysis, the average spectral counts were used for each protein at all three time-points for patients from each group. The log 10 of the average spectral count for each protein in the control group was then calculated and plotted against the log 10 of the average spectral count for each protein in the diseased group for time-points 1 and 8, FIGS. 4a and 4b, respectively. Proteins falling above the upper red-dashed line are proteins that are elevated in the diseased group and proteins falling below the lower red-dashed line are proteins that are elevated in the control group. Proteins falling between the two red dashed lines are not significantly different between the two groups, although proteins in this area may still be of interest upon further evaluation.

Looking at FIG. 4A, there are not many proteins that fall outside the dashed lines, which can be expected since this is the baseline time-point. However, when looking at FIG. 4b the number of proteins that are outside the dashed lines increases dramatically. There are three proteins that are increased in the diseased group at time-point 8 that are considered of “proteins of high interest” and they are proteins 7, 8, and 31, which correspond to annexin A2, plakoglobin, and serpin B3, respectively. These proteins appear in boldface in Table 1. The proteins that are decreased in the diseased group at time-point 8 are also proteins of interest and they appear in italics in Table 1. Proteins that are not in boldface or italics are not excluded from further investigation and may be of importance. In particular, proteins 1, 3, and 6 are of interest because they have been seen free in serum and the ratio of free vs bound for these proteins, as well as for any of the other proteins listed, may be indicative of the disease process. Ultimately, any protein listed in the supplemental table may be a protein that could have potential clinical use.

The three proteins of “high interest” are particularly intriguing because they are implicated in known diseases and are elevated in diseased patients at time-point 8. Plakoglobin is intriguing because it is a component of the desmosomes, which are major intracellular adhesive junctions that anchor intermediate filaments to the plasma membrane (Green et al. Nature Reviews Molecular Cell Biology 2000, 1:208-216). Mutations in genes encoding for cardiac desmosomal proteins is prevalent in patients with arrhythmogenic right ventricular dysplasia/cardiomyopathy (ARVD/C), an inherited heart disease that is clinically defined by the presence of particular electrical, functional, and structural right ventricular abnormalities and histologically by replacement of cardiomyocytes with fibrous or fibrofatty tissue (Basso et al. Lancet 2009, 373:1289-1300; McKenna et al. British Heart Journal 1994, 71:215-218). Work over the past decade has shown that ARVC is an autosomal dominant trait frequently caused by mutations in genes that encode important structural proteins found within the desmosome (Awad et al. Nat Clin Pract Cardiovasc Med 2008, 5:258-267). Recent work has shown that mutations in the genes encoding for desmosomal proteins are also prevalent in patients with dilated cardiomyopathy (Elliott et al. Circulation Cardiovascular Genetics 2010, 3:314-322).

The fact that an increase is observed in the amount of plakoglobin bound to the ABPPC in diseased patients indicates that there is degradation of the desmosomes in these patients and therefore loss of structural integrity of the cell-cell interactions within the myocardium, which is highly probable since the patients in this group are showing elevated levels of cTnI. Albumin could be serving as a sponge to bind these proteins that are released from degraded desmosomes. If this is the case and these and other desmosomal proteins, such as the plakophilins, desmogleins, and desmocollins (all of which are represented in the ABPPC) would be elevated in the ABPPC as a result of myocardial ischemia, then it stands to reason that they would also be elevated in the ABPPC for patients with other cardiac disorders and could be used as powerful biomarkers in cardiovascular medicine.

SERPINB3 is a peptidase inhibitor that is implicated in the survival of squamous carcinoma cells (Ahmed et al. Biochem Biophys Res Commun 2009, 378:821-825) and in chronic liver disease through its modulation of TGF-β (Turato et al. Laboratory Investigation 2010, 90:1016-1023). Annexin A2 is a member of the annexin family, which is a family of calcium-dependent phospholipid-binding proteins that play a role in the regulation of cellular growth and in signal transduction pathways. Annexins have been shown to be involved in a variety of cellular processes, including trafficking and organization of vesicles, exo- and endocytosis, and in calcium ion channel formation (Gerke et al. Nat Rev Mol Cell Biol 2005, 6:449-461) and annexin A2 has been proposed as a differential diagnostic marker of hepatocellular tumors (Ji et al. Inter J Mol Med 2009, 24:765-771; Longrich et al. Pathol Res Pract 2010, Article in Press doi:10.1016/j.prp.2010.09.007). The implication of the free form of these proteins in disease makes the fact that they are observed in the ABPPC very intriguing and the ABPPC bound forms of these proteins (or any of the proteins observed in the ABPPC) could have significant diagnostic potential.

We claim:

1. A method of diagnosing ischemia in a subject, comprising
(a) determining the level of at least one biomarker selected from the listing in Table 2 in a biological sample obtained from said subject, wherein said biomarker comprises an albumin-bound protein/peptide complex (ABPPC), and
(b) quantifying the level determined in the biological sample to a control level in a normal subject population, wherein an increase or decrease in the level, compared to control level, is indicative of ischemia.

2. The method of claim 1 that is a diagnostic assay.

3. The method of claim 1 that is a prognostic or monitoring assay.

4. The method of claim 1 wherein ischemia is selected from the group consisting of myocardial ischemia, organ ischemia, renal ischemia and brain ischemia.

5. The method of claim 1, wherein the ABPPC is selected from the group consisting of annexin A2, plakoglobin and serpinB3.

6. The method of claim 1, wherein the subject sample is derived from blood, plasma or body fluids.

7. The method of claim 1, wherein the biomarker(s) are detected using mass spectrometry.

8. The method of claim 1, wherein the biomarker(s) are detected using SEC, HPLC, affinity chromatography, gel methods and/or immunoassay.

9. The method of claim 1, wherein the subject is a mammal.

10. The method of claim 9, wherein the subject is a human.

11. A diagnostic or prognostic kit comprising
an antibody or a chemical moiety to specifically capture or enrich albumin in a biological sample;
a secondary antibody or chemical moiety to one or more specific modified or unmodified proteins or peptides bound to albumin selected from the listing of Table 2; and
at least one component for detection and/or quantification of the amount of secondary antibody bound.

12. The kit of claim 11 comprising a plurality of secondary antibodies.

13. A mass spectroscopy kit comprising
at least one antibody directed against a protein or protein fragment selected from the listing of Table 2; and
a mass spectroscopy labeled internal protein standard.
